(19) United States
(12) Patent Application Publication
(10) Pub. No.: US 2023/0405156 A1
(43) Pub. Date: Dec. 21, 2023

(54) METHODS OF DIAGNOSING AND PROGNOSING CANCERS OF THE HEAD AND NECK

(71) Applicant: The Board of Trustees of the Leland Stanford Junior University, Stanford, CA (US)

(72) Inventor: Matt Lechner, Stanford, CA (US)

(21) Appl. No.: 17/816,317

(22) Filed: Jul. 29, 2022

Related U.S. Application Data
(60) Provisional application No. 63/227,783, filed on Jul. 30, 2021.

(51) Int. Cl. A61K 51/04; G01N 33/574; A61K 31/555; A61K 31/7068; A61K 33/243

(52) U.S. Cl. CPC A61K 51/0482; G01N 33/57488; A61K 31/555; A61K 31/7068; A61K 33/243; A61K 2123/00; A61K 2121/00

(57) ABSTRACT

Methods are provided for diagnosing, prognosing, and treating cancers expressing SSTR2, in particular cancers of the head and neck, such as skull base malignancies, head and neck cancers and brain cancers, such as nasopharyngeal cancer and olfactory neuroblastoma. Provided is a non-invasive imaging method, where an imaging agent is administered to an individual with a tumor; where said imaging agent is then quantified within the tumor using PET-CT imaging to provide an indirect measurement of somatostatin receptor expression and to serve as a prognostic biomarker and companion diagnostic biomarker for treatment stratification in sinonasal cancers. Also provided are methods to directly measure SSTR2 expression from biopsied tumors. These methods robustly identify which patients will have increased rates of survival based on indirect and/or direct measurements of the level of expression of somatostatin receptors in tumors. In some embodiments, prognostic power of the non-invasive imaging method can be further increased by utilizing immunohistochemical analysis of tumor biopsies. In some embodiments, the methods further comprise selecting a treatment regimen for the individual based on the analysis.

### CROSS-REFERENCE TO RELATED APPLICATIONS

This application claims the benefit of U.S. Provisional Patent Application No. 63/227,783, filed Jul. 30, 2021, which application is incorporated herein by reference in its entirety.

### INCORPORATION BY REFERENCE OF SEQUENCE LISTING

A Sequence Listing is provided herewith as a Sequence Listing XML, UCLB-001_S21-203_Sequence_Listing created on Sep. 11, 2023, and having a size of 17,847 bytes. The contents of the Sequence Listing XML are incorporated herein by reference in their entirety.

### BACKGROUND

Brain cancers and other cancers of the skull base and head and neck region are extremely challenging malignancies, with significant morbidity and mortality (10th leading cause of death for men and women). The last two decades have showed some progress for brain tumor researchers, heralded by the adoption of one drug (Temozolomide) into standard-of-care of glioblastoma and the emergence of novel therapeutic agents such as DNA damage repair inhibitors, with promising early data. There remains a critical need to develop entirely new strategies for the treatment of this difficult-to-treat disease. The heterogenous and challenging biology of brain cancers means that multiple approaches may be necessary.

Nasopharyngeal cancer (NPC) is a malignant epithelial tumor showing squamous differentiation. It occurs most frequently in the pharyngeal recess (fossa of Rosenmuller), an area that is difficult to access surgically due to the anatomical constraints in creating open access for surgical resection. NPC is classified into three histological subtypes by the World Health Organization (WHO I-Ill). Non-keratinizing NPC subtypes (WHO II-Ill) are strongly related to EBV infection, a known risk factor for NPC. Other predisposing genetic and environmental factors have resulted in a striking geographical distribution of NPC. Treatment in the early stages of disease comprises radiotherapy and, more rarely, surgery, both of which can result in considerable morbidity. Systemic treatment using platinum based therapies is reserved for the recurrent and metastatic settings where the median survival ranges between 12 and 24 months.

Somatostatin receptor (SSTR) is a G protein-coupled cell surface receptor whose activation by extracellular ligands leads to inhibition of cell proliferation. In neuroendocrine tumors (NETs), SSTR2 expression is imaged with ⁶⁸Ga-dodecane tetraacetic acid (DOTA)-peptide radioconjugates and therapeutically exploited by ¹⁷⁷Lu- or ⁹⁰Y-DOTA-peptide radioconjugates and SSTR2 agonists such as the octreotide octapeptide. In NETs, SSTR2 receptor activation is associated with a decrease in proliferation, with SSTR2 agonists being cytostatic and effective at controlling growth but only in tumors with proliferation rates below 10%. A previous study in five patients with primary EBV-positive NPC showed increased uptake of ⁶⁸Ga-DOTA- (Tyr³)-octreotide or edotreotide (TOC), with two case reports published around the same time, indicating the candidacy of SSTR expression for functional imaging of NPC. In a study of 12 NPCs, SSTR autoradiography on tissue samples confirms these unexpected imaging results. The ability of SSTR2 agonists to control NPC tumor growth is unknown.

The present disclosure provides improved methods for diagnosing, prognosing, in addition to selecting treatment regimens for cancers of the head and neck expressing SSTR2.

### SUMMARY

Methods are provided for diagnosing, prognosing, and treating cancers expressing SSTR2, in particular cancers of the head and neck, such as skull base malignancies, head and neck cancers and brain cancers. In an embodiment, provided is a non-invasive imaging method, where an SSTR2 imaging agent is administered to an individual with a suspected cancer; where the SSTR2 imaging agent is then quantified on cells within the suspected tumor using PET-CT imaging, to provide an indirect measurement of SSTR2 expression and to serve as a prognostic biomarker and companion diagnostic biomarker for treatment stratification of cancers expressing SSTR2. Also provided are methods to directly measure somatostatin receptor expression on cells from biopsied tumors, e.g. by immunohistochemistry (IHC), including a grading system; and including a computer-based automated analysis.

The methods disclosed herein robustly identify which patients will have increased rates of survival based on indirect and/or direct measurements of the level of expression of SSTR2 on tumor cells, e.g. nasopharyngeal cancer expressing SSTR2. In some embodiments, the prognostic power of the non-invasive imaging method is further increased by utilizing immunohistochemical analysis of tumor biopsies. A significant correlation of SSTR2 expression levels with in vivo uptake of ⁶⁸Ga-DOTA peptides, demonstrating the utility of this imaging modality as a noninvasive marker to monitor SSTR2 expression and as a target for SSTR2 receptor-targeted therapy. In some embodiments, an individual is classified by EBV and SSTR2 status, where the poorest prognosis is for those patients who are both EBV negative and SSTR2 negative. In some embodiments, the methods further comprise selecting a treatment regimen for the individual based on the analysis, and treating the patient accordingly. In some embodiments an individual is selected for SSTR2 receptor-targeted treatment when the tumor uptake score is 2, or 3.

An individual selected for assessment by the method of the invention has or is suspected to have, a cancers of the head and neck, such as skull base malignancies, head and neck cancers and brain cancers expressing SSTR2. In some embodiments the individual has or is suspected to have, nasopharyngeal cancer or sinonasal cancer expressing SSTR2. In some embodiments the individual has been previously diagnosed with nasopharyngeal cancer or sinonasal cancer. In some embodiments the sinonasal cancer is, without limitation, olfactory neuroblastoma, squamous cell carcinoma, adenocarcinoma, adenoid cystic carcinoma, sinonasal undifferentiated carcinoma, etc., or is nasopharyngeal cancer. In some embodiments, the cancer is a nasopharyngeal cancer. In some embodiments the nasopharyngeal cancer is a non-keratinizing histological subtype (WHO II-Ill). In some embodiments, the cancer is local, recurrent, or metastatic cancer. In some embodiments, the cancer is olfactory neuroblastoma.

In individuals with a sinonasal cancer, it is shown herein that increased SSTR2 expression is associated with a positive response to anti-somatostatin receptor treatment. Somatostatin receptor expression level can be determined through immunohistochemical analysis of biopsied tumors or through analysis of an imaging agent uptake into tumor tissues. Imaging agents that find use in the present disclosure, include without limitation, ⁶⁸Ga-dodecane tetraacetic acid (DOTA)-(Tyr³)-octreotide or edotreotide (TOC), ⁶⁸Ga-DOTA-Nal3-octreotide (NOC), ⁶⁸Ga-DOTA-(Tyr³)-octreotate (TATE), etc. Immunohistochemical analysis of tumor biopsies may be scored based on the intensity of staining of SSTR2, where a score of 1 corresponds to weak staining not easily seen via low power objectives, a score of 2 corresponds to moderate staining still seen on a low power objective and a score of 3 corresponds to strong staining easily visible via a low power objective with a score of 2 or 3 correlating with a positive prognosis in response to anti-somatostatin receptor treatment. Analysis of a labeled peptide uptake into tumor tissues may be scored based on the maximized standardized uptake value (SUVmax) where a SUVₘₐₓ score of at least about 8, at least about 10, or more is considered to be a high density of somatostatin receptor, i.e. SSTR2 positive.

The methods disclosed herein include steps of data analysis, which may be provided as a program of instructions executable by computer and performed by means of software components loaded into the computer. Such methods include, for example, an automated analysis of immunohistochemical staining of tumor samples. An image of a stained sample deconvolved to digitally separate hematoxylin stains. Regions of tissue, white space, and artefact are manually annotated on a subset of sections, which was then used to train a tissue classifier. A deep-learning algorithm is used to detect cell nuclei and make cell level measurements. An object classifier is then trained on manually annotated areas of tumor and stroma and integrated into the pipeline to determine extent of staining in the respective regions. The output of the analysis includes the number of cell detections and H-score, where the H-score is the sum of the percentage of cells stained, multiplied by the degree of intensity, yielding scores within a range of 0 and 300. The H-score provides a measure of SSTR2 staining on the tumor, and provides prognostic classification, which can be used in determining appropriate treatment for the patient. The method may further comprise providing a computer-generated report comprising the prognosis of the condition.

Methods of the present disclosure can be used to facilitate personalized selection of treatment for patients with a number of different cancers. In some embodiments, types of sinonasal cancers that may be suitable for analysis using methods of the present disclosure include, but are not limited to, nasopharyngeal cancer, olfactory neuroblastoma, squamous cell carcinoma, adenocarcinoma, adenoid cystic carcinoma, and sinonasal undifferentiated carcinoma. In one embodiment, the cancer is a nasopharyngeal cancer or an olfactory neuroblastoma. An individual with a high score from IHC analysis and/or peptide uptake analysis, can be selected, and treated, with an SSTR2 therapeutic agent, which may be used in combination with additional therapeutic agents. SSTR2 therapeutic agents of interest include, without limitation, drug and radionuclide conjugates of somatostatin analogs and other SSTR2 binding agents, e.g. PEN-221, ¹⁷⁷Lu conjugates, ⁹⁰Y conjugates, drug conjugates, etc. An individual with a low score from IHC analysis or ⁶⁸Ga-DOTA peptide uptake analysis can be selected, and treated, with a non-SSTR2 agent, e.g. chemotherapy, immunotherapy such as an immune checkpoint inhibitor (ICI), radiation therapy, and the like. In some embodiments, an individual with a low score from IHC analysis or peptide uptake analysis is treated with gemcitabine and cisplatin.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1E. Evaluation of SSTR2 expression in a multi-institutional clinical cohort of NPC. a Anatomical localization and representative images of hematoxylin and eosin (H&E) stained histology, somatostatin receptor 2 (SSTR2) expression assessed by immunohistochemistry (IHC) and Epstein-Barr Virus (EBV)-encoded small RNAs (EBER) assessed by in situ histochemistry). b Beanplot of the SSTR2 IHC score in EBV-positive (n=278) and EBV negative (n=60) cases of NPC (W=3085.5, p=4.0e-14, Wilcoxon two-sided test). c Heatmap representation of the clinical annotations in relation to SSTR2 expression levels (asterisks indicate significant associations with SSTR2 expression using multivariate analysis). d, e SSTR2 status and EBV status were not statistically different in the primary (n=37), local recurrent (n=47), and metastatic (n=20) tumor tissue (images of rNPC42; ×400; scale bar 25 μm and summary of data; two-sided Fisher's test on proportion of SSTR2 positivity in primary, metastatic and local recurrence samples; p=0.32). Representative tumor samples from a single patient are shown in d. Source Data are provided as a Source data file.

FIGS. 2A-2K. EBV infection is associated with SSTR2 upregulation in NPC—in vitro experiments and external validation dataset. a Immunofluorescence analysis of EBNA1 (red) and SSTR2 (green) expression in two representative examples of cultured primary respiratory epithelial cells before and after infection with the epitheliotropic M81 EBV strain. Nuclei are counterstained with DAPI (blue); Replication (n=5). b Percentages of cells positive for EBNA1 or co-expression of SSTR2 and EBNA1 c LMP1 induces SSTR2 expression in NP69, an immortalized normal nasopharyngeal epithelial cell line (relative quantities of SSTR2 were calculated using the comparative threshold cycle method and normalized using human beta-actin as endogenous control. Data is presented as a ratio relative to vector control). d LMP1-mediated SSTR2 induction in NP69 cells is inhibited by ectopic expression of TRAF3, a negative regulator of NF-κB. e In NP69 cells, LMP1-induced SSTR2 expression is suppressed by the NF-κB inhibitor BAY 11-7085 and MEK inhibitor U0126. f Both CTAR1 and CTAR2 regions of LMP1 (FIG. 6) are essential for LMP1-mediated SSTR2 induction. LMP1 mutant constructs 3 A, Delta 8 C and 3A+Delta 8 C target CTAR1, CTAR2, and both regions respectively. g SSTR2 expression by LMP1 is dose dependent. h In C666-1 cells, LMP1-induced SSTR2 expression is suppressed by the NF-κB inhibitor BAY 11-7085 and MEK inhibitor U0126. i siRNAs mediated knockdown of the subunits of activated NF-κB signal complexes, NFκB1 (p105/p50), RELB, NFκB2 (p100/p52), or c-Jun in C666-1, both resulted in significant SSTR2 suppression. j Left panel, PCA on independent RNA-seq data of NPC (n=113) identifies two groups of NPC tumors. The color of the samples is based on unsupervised hierarchical clustering. Middle panel, Differential gene expression analysis between Group 1 and 2 tumors shows that SSTR2 is highly-expressed in Group 1 tumors (log 2 fold change=2.3, adjusted p=2.7e-32). Right panel, Pathway analysis demonstrates that Group 1 tumor show significant enrichment of viral biogenesis pathways. k Left panel, Heatmap of gene expression of EBV genes in an independent cohort of NPC (tumor n=31, normal n=10). Middle panel, Microarray SSTR2 expression is positively correlated with viral LMP1 expression (W=129, p=0.041, Wilcoxon two-sided test). Center line displays the median, boxes display the interquartile range. Whiskers display 1.5× the interquartile range. Outliers lie beyond the whiskers. Right panel, Pathway analysis demonstrates that LMP1-expressing tumor samples are enriched in viral biogenesis pathways. *, **, ***, and **** denote a significant difference between groups of P<0.05, P<0.01, P<0.0001, and P<0.0001, respectively. Source Data are provided as a Source data file.

FIGS. 3A-3F. In vitro and in vivo effects of SSTR2 agonists on the C666-1 NPC cell line. a Immunohistochemical characterization of C666-1, NPC43, and C17 cell lines cultured in vitro and from xenografted C666-1, C15, C17, or C18 tumor tissues (scale bar 100 μm); Replication n=2. b In vitro dose response curves and half-maximal effective concentration (EC50 values) of the indicated SSTR agonists on C666-1, NPC43, and C17 cells. EC50=half-maximal effective concentration. c Growth curves of C666-1 tumors in nude mice treated with vehicle (n=9), octreotide (n=9), or PEN-221 (n=8). The dotted lines indicate the time points of drug injection. d Kaplan-Meier curves of athymic nude mice with C666-1 tumors, treated with vehicle control (n=9), octreotide (n=9), or PEN-221 (n=8), with dotted lines showing time points of drug or vehicle injection (*p=0.0368; two-sided Log-rank Mantel-Cox test). e Geneset enrichment analysis reveals upregulation of senescence pathways 24 h post lanreotide treatment (left), and upregulation of apoptosis and mitotic spindle assembly pathways 24 h post-PEN-221 treatment (right) in treated vs untreated cell lines. f mRNA sequencing analysis of C666-1 cells treated in vitro (72 h with PEN-221) reveals downregulation of SSTR2 expression (two-sided Wald test, adjusted p=3.9e-7). Center line displays the median, boxes display the interquartile range. Whiskers display 1.5× the interquartile range. Source Data are provided as a Source data file.

FIGS. 4A-4E. Visualization and prognostic value of SSTR2 expression in NPC patients. a Visualization of SSTR2 expression by ⁶⁸Ga-DOTA-TATE PET-CT imaging (clinical characteristics and SSTR2 status of NPC patients undergoing ⁶⁸Ga-DOTA-TATE PET-CT imaging are shown in Table 5). b Correlation of SSTR2 expression with in vivo uptake of ⁶⁸Ga-DOTA-TATE. SSTR2 IHC score shows significant positive correlation to SUVmax of biopsied lesion. (black circles: biopsied lesions, n=12; Spearman's correlation coefficient: Rs=0.65; p=0.023). d SSTR2 expression status remains prognostic independent of EBV status, age, and primary tumor (T), lymph node (N), and metastasis (M) staging. n.s not significant; *p<0.05; **p<0.01; ***p<0.001. Vertical lines display the hazard ratio estimate, horizontal lines display the 95% confidence interval. e Proposed model of NPC oncogenesis and cancer progression involving EBV and SSTR2 expression. In the multistep carcinogenesis of NPC, inactivation of tumor suppressor genes is believed to occur prior to EBV infection and to be induced by dietary carcinogens and other environmental factors. Infection of nasopharyngeal cells with EBV and establishment of a latent infection probably occurs at a late stage in the acquisition of the malignant phenotype. Genetic alterations identified in premalignant nasopharyngeal epithelium may play crucial roles to support stable EBV infection. Once a premalignant cell has been infected by EBV, it appears to rapidly evolve towards an invasive tumor, with the stage of EBV-positive in situ carcinoma being very transient. SSTR2 expression is acquired following the onset of latent EBV infection by LMP1 expression via NF-κB signaling. On the basis of this tentative scenario, pharmacological agonists of SSTR2 are expected to provide the maximal benefit for three types of indications: (1) as part of the initial curative treatment of the primary tumors, (2) as part of adjuvant treatment following clinical remission of the primary tumor; (3) with a prophylactic intent for subjects at risk of NPC manifested by EBV serological changes and/or increasing circulating EBV DNA load. Source Data are provided as a Source data file.

FIG. 5. RT-PCR analysis of EBV-infected primary epithelial cells using EBER-specific primers and PCR with SSTR2-specific primers confirmed EBV infection and induction of SSTR2 transcription in infected epithelial cells. The figure shows the amplification product (425 bp) after PCR of cells infected or not by the virus. The graph shows the EBER expression levels in infected cells relative to uninfected epithelial cells after normalization with GAPDH signals (ΔΔCT); Replication n=2;

FIGS. 6A-6D. LMP1 induces SSTR2 expression in EBV-infected nasopharyngeal epithelial cells. (A) Transient transfection of LMP1, but not other EBV latent genes, EBNA1 and LMP2A induces SSTR2 expression in NP69 nasopharyngeal epithelial cells. (B) In LMP1-expressing NP69 and C666-1 cells, SSTR2 expression was not suppressed by AKT inhibitor treatment. (C) siRNAs mediated knockdown of the subunits of activated NF-κB signal complexes, NFκB1 (p105/p50), NFκB2 (p100/p52), RelB, RelA or BCL3 in C666-1 NPC cells. Significant SSTR2 suppression was shown in NPC cells treated with NFκB1, NFκB2 and RelB siRNAs. (D) LMP1 mediates nuclear accumulation of NF-κB subunits and induces SSTR2 expression.

FIG. 7. Proposed signal transduction pathways downstream of the EBV LMP1, leading to SSTR2 expression.

FIGS. 8A-8B. A) LMP1 and SSTR2 IHC staining (brown) in EBV related tumors. Lymphoepithelioma-like carcinoma of the lung (LELC) shows strong diffuse staining of SSTR2 and LMP1. In EBV-positive Hodgkin's Lymphoma (HL), LMP1 and SSTR2 staining is shown in the malignant EBV-positive Reed-Sternberg cells, surrounded by inflammatory infiltrate. EBV-associated Smooth Muscle tumor (EBV SM) is absent for both LMP1 and SSTR2. B) High-resolution images (×400) of two cases of Hodgkin's Lymphoma (HL) with strong LMP1 and SSTR2 staining shown in the malignant EBV-positive Reed-Sternberg cells.

FIG. 9. Positive correlation between SSTR2 and NFkB1 expression (linear regression: bb1=0.78, rr2=0.31, two-sided t-test: p=0.0001), however, no association between NFkB and LMP1 was found in this dataset (data not shown).

FIG. 10. Kaplan-Meier curves for European center patients jointly classified by their EBV status and SSTR2 status.

FIGS. 11A-11B. A) Showing a piechart of the antibodies used: UMB-1 antibody alone was used in 273 cases (68.1%) while SS-8009-RM antibody alone was used in 28 cases (7%). Both antibodies were used in 100 cases (24.9%) with an overall moderate inter-rater reliability (κ=0.49), but a substantial agreement in the 67 cases where tissue samples were available (κ=0.755). Staining in the TMA (n=33) group showed only a slight agreement (κ=0.183) B) Showing two exemplary cases where both antibodies were used in tissue samples (left: UMB1, right: SS-8000-RM). Staining was performed one time with each antibody using positive controls.

FIGS. 12A-12D. Clinical characteristics of olfactory neuroblastoma. A) Anatomical localization and representative images of histology (H&E staining), expression of common markers (S100, chromogranin A, synaptophysin) and SSTR2, which were assessed by immunohistochemistry; B) Heatmap representation of clinical annotations; C) Bar graph representation of common symptoms at presentation; D) Kaplan-Meier overall survival of primary cases.

FIGS. 13A-13E. Confirmation of SSTR2 expression in local recurrences and metastases and clinical trial on Somatostatin receptor (SSTR) 2-positive olfactory neuroblastoma. A) Representative images of SSTR2 expression, with corresponding haematoxylin and eosin (H&E), in local recurrence and lymph node metastasis, determined by immunohistochemistry (IHC). B-D) Immunohistochemical characterization of tumor biopsies (SSTR2 and Chromogranin correlation of SSTR2 IHC with in vivo uptake of ⁶⁸Ga-DOTATATE in PET MRI imaging of three patients who were enrolled in the LUTHREE trial (NCT03454763) and underwent SSTR2-targeted peptide-radionuclide receptor therapy (PRRT). Pre-treatment ⁶⁸Ga-DOTATATE positron emission tomography (PETs) MRI with corresponding magnetic resonance imaging (MRI) and subsequent MRI 1 year post treatment.

FIG. 14. Expression data on SSTR2 in ONB. 82.4% of the cohort, for which immunohistochemical staining for SSTR2 were available, were positive for the biomarker.

FIG. 15. Workflow of the development of an automated digital pathology pipeline for the high through-put evaluation of SSTR2 staining.

FIG. 16. Positive SSTR2 expression in brain cancers. Representative image of SSTR2 expression in astrocytoma and oligodendroglioma.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

SSTR2. Somatostatin receptor type 2 belongs to the G-protein coupled receptor family. It is a receptor for somatostatin-14 and -28. Somatostatin-14 and -28 work by binding to the receptor with the help of a G-protein. This inhibits adenylyl cyclase and calcium channels. Somatostatin 2 receptors have been found in concentration on the surface of tumor cells, particularly those associated with the neuroendocrine system where the overexpression of somatostatin can lead to many complications. The reference sequence of the human protein may be accessed at Genbank, NP_001041.

Somatostatin. In addition to the native ligand somatostatin, somatostatin analogs are known in the art, including, for example, octreotide, DOTA-TATE, DOTA-NOC, DOTA-TOC, depreotide, pasireotide, lanreotide, etc. Octreotide has been successfully used in combination with radio-peptide tracers to locate adrenal gland tumors through scintigraphic imaging. Octreotide and other analogs are preferred for this use due to their extended half-life compared to the naturally-occurring hormone.

Radiographic moieties for use as imaging agents in the present invention include compounds and chelates with relatively large atoms, such as gold, iridium, technetium, barium, thallium, iodine, and their isotopes. Positron emitting moieties for use in the present invention include, without limitation, ¹⁸F, ⁶⁴Cu and ⁶⁸G. The affibody may be conjugated to a chelating agent, e.g. 1,4,7,10-tetraazacyclododecane-N,N′,N″,N′″-tetraacetic acid (DOTA), for chelating a radioisotope. Among the most commonly used positron-emitting nuclides in PET are included ⁶⁸Ga, ¹¹C, ¹³N, ¹⁵O, and ¹⁸F. For example, ⁶⁸Ga-labeled 1,4,7,10-tetraazacyclododecane-N,N9,N99,N999 tetraacetic acid-D-Phe1-Tyr3-octreotide (DOTA-TOC) can be used for PET in the methods of the present disclosure. Other SSTR2 tracers include, for example, ⁶⁸Ga-DOTA-Tyr3-octreotate (68Ga-DOTA-TATE) and ⁶⁸Ga-DOTA-1-Nal3-octreotide (68Ga-DOTANOC).

Optically visible moieties for use as imaging moieties include fluorescent dyes, or visible-spectrum dyes, visible particles, and other visible labeling moieties. Fluorescent dyes such as fluorescein, coumarin, rhodamine, bodipy Texas red, and cyanine dyes such as indocyanine green, are useful when sufficient excitation energy can be provided to the site to be inspected visually. Endoscopic visualization procedures may be more compatible with the use of such labels. For many procedures where imaging agents are useful, such as during an operation to resect a brain tumor, visible spectrum dyes are preferred. Acceptable dyes include FDA-approved food dyes and colors, which are non-toxic, although pharmaceutically acceptable dyes which have been approved for internal administration are preferred.

SSTR therapeutic agents include a number of cytotoxic agents that target SSTR2 expressing cells. Suitable SSTR2 agents, include without limitation, conjugates of somatostatin analogs such as octreotide, DOTA-TATE, DOTA-NOC, DOTA-TOC, depreotide, pasireotide, lanreotide, etc. or other SSTR2 ligands, conjugated to a radionuclide or cytotoxic agent. Preferred radionuclides for use as cytotoxic moieties are radionuclides that are suitable for pharmacological administration. Such radionuclides include, for example, ⁹⁰Y, ¹⁷⁷Lu, ¹²³I, ¹²⁵I, ¹³¹I, ²¹¹At, ⁶⁷Cu, ¹⁸⁶Re, ¹⁸⁸Re, ²¹²Pb, ²¹²Bi, etc., e.g. lutetium-177 or Ytrium-90 SSTR2 ligands.

Peptide receptor radionuclide therapy (PRRT) finds use in the present disclosure. PRRT is part of the wider concept of targeted radionuclide therapy. PRRT delivers destructive radiation to cancer cells via radiolabeled peptides able to bind specifically to peptide receptors expressed in higher density on the tumor cell membrane than in nontumor tissues. This is the case for many G-protein-coupled receptors, whose overexpression is linked to numerous human malignancies. Regulatory peptides targeting G-protein-coupled receptors are a rich source of vectors that can be chemically tuned to transport radioactivity while preserving their receptor affinity. PRRT is used in the treatment of metastatic or unresectable cancers through systemic or, more occasionally, locoregional administration.

Peptide-based radiotherapeutics require a chelator that stably chelates the radiometal in vivo in order to exclude deposition of free radiometal in normal tissues. The conjugation of a universal chelator, such as DOTA, allows radiolabeling with different radiometals sharing similar chemical properties, such as the trivalent radiometals ⁹⁰Y, ¹⁷⁷Lu, ²²⁵Ac, or ⁶⁸Ga (used in PET). This versatility enabled the development of radiotheranostics, which are essential for selecting patients who are suitable for PRRT. Such an approach may provide more efficient and personalized patient care, because properties of the vector, type of radionuclide, and dose of radioactivity can be tailored to address specific clinical needs.

Cytotoxins suitable for use with the methods described herein include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as .alpha.-amanitin, and derivatives thereof), and agents capable of disrupting protein biosynthesis (e.g., agents that exhibit rRNA N-glycosidase activity, such as saporin and ricin A-chain), among others known in the art. For example, PEN-221 is a conjugate consisting of microtubule-targeting agent DM1 linked to the C-terminal side chain of Tyr3-octreotate. Other peptides suitable for use as cytotoxic agents include, for example, ⁹⁰Y-DOTA-TOC or ¹⁷⁷Lu-DOTA-octreotate, ¹⁷⁷Lu-DOTA-TATE. One of skill in the art can select a suitable radionuclide or cytotoxin for this purpose.

In some embodiments, the cytotoxin is a microtubule-binding agent (for instance, maytansine or a maytansinoid), an amatoxin, pseudomonas exotoxin A, deBouganin, diphtheria toxin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, or a variant thereof, or another cytotoxic compound described herein or known in the art. For example, a cytotoxin may be a maytansinoid selected from DM1, and DM4; an auristatin selected from monomethyl auristatin E and monomethyl auristatin F; an anthracycline selected from daunorubicin, doxorubicin, epirubicin, and idarubicin; etc.

Additional cytotoxins suitable for use with the compositions and methods described herein include, without limitation, 5-ethynyluracil, abiraterone, acylfulvene, adecypenol, adozelesin, aldesleukin, altretamine, ambamustine, amidox, amifostine, aminolevulinic acid, amrubicin, amsacrine, anagrelide, anastrozole, andrographolide, angiogenesis inhibitors, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, asulacrine, atamestane, atrimustine, axinastatin 1, axinastatin 2, axinastatin 3, azasetron, azatoxin, azatyrosine, baccatin Ill derivatives, balanol, batimastat, BCR/ABL antagonists, benzochlorins, benzoylstaurosporine, beta lactam derivatives, beta-alethine, betaclamycin B, betulinic acid, bFGF inhibitors, bicalutamide, bisantrene, bisaziridinylspermine, bisnafide, bistratene A, bizelesin, breflate, bleomycin A2, bleomycin B2, bropirimine, budotitane, buthionine sulfoximine, calcipotriol, calphostin C, camptothecin derivatives (e.g., 10-hydroxy-camptothecin), capecitabine, carboxamide-amino-triazole, carboxyamidotriazole, carzelesin, casein kinase inhibitors, castanospermine, cecropin B, cetrorelix, chlorins, chloroquinoxaline sulfonamide, cicaprost, cis-porphyrin, cladribine, clomifene and analogues thereof, clotrimazole, collismycin A, collismycin B, combretastatin A4, combretastatin analogues, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, 2′deoxycoformycin (DCF), deslorelin, dexifosfamide, dexrazoxane, dexverapamil, diaziquone, didemnin B, didox, diethylnorspermine, dihydro-5-azacytidine, dihydrotaxol, dioxamycin, diphenyl spiromustine, discodermolide, docosanol, dolasetron, doxifluridine, droloxifene, dronabinol, duocarmycin SA, ebselen, ecomustine, edelfosine, edrecolomab, eflornithine, elemene, emitefur, epothilones, epithilones, epristeride, estramustine and analogues thereof, etoposide, etoposide 4′-phosphate (also referred to as etopofos), exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, homoharringtonine (HHT), hypericin, ibandronic acid, idoxifene, idramantone, ilmofosine, ilomastat, imidazoacridones, imiquimod, immunostimulant peptides, iobenguane, iododoxorubicin, ipomeanol, irinotecan, iroplact, irsogladine, isobengazole, jasplakinolide, kahalalide F, lamellarin-N triacetate, lanreotide, leinamycin, lenograstim, lentinan sulfate, leptolstatin, letrozole, lipophilic platinum compounds, lissoclinamide 7, lobaplatin, lometrexol, lonidamine, losoxantrone, loxoribine, lurtotecan, lutetium texaphyrin, lysofylline, masoprocol, maspin, matrix metalloproteinase inhibitors, menogaril, merbarone, meterelin, methioninase, metoclopramide, MIF inhibitor, ifepristone, miltefosine, mirimostim, mithracin, mitoguazone, mitolactol, mitomycin and analogues thereof, mitonafide, mitoxantrone, mofarotene, molgramostim, mycaperoxide B, myriaporone, N-acetyldinaline, N-substituted benzamides, nafarelin, nagrestip, napavin, naphterpin, nartograstim, nedaplatin, nemorubicin, neridronic acid, nilutamide, nisamycin, nitrullyn, octreotide, okicenone, onapristone, ondansetron, oracin, ormaplatin, oxaliplatin, oxaunomycin, paclitaxel and analogues thereof, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, phenazinomycin, picibanil, pirarubicin, piritrexim, podophyllotoxin, porfiromycin, purine nucleoside phosphorylase inhibitors, raltitrexed, rhizoxin, rogletimide, rohitukine, rubiginone B1, ruboxyl, safingol, saintopin, sarcophytol A, sargramostim, sobuzoxane, sonermin, sparfosic acid, spicamycin D, spiromustine, stipiamide, sulfinosine, tallimustine, tegafur, temozolomide, teniposide, thaliblastine, thiocoraline, tirapazamine, topotecan, topsentin, triciribine, trimetrexate, veramine, vinorelbine, vinxaltine, vorozole, zeniplatin, and zilascorb, among others.

The terms “biomarker,” “biomarkers,” “marker” or “markers” for the purposes of the invention refer to, without limitation, proteins together with their related metabolites, mutations, variants, polymorphisms, modifications, fragments, subunits, degradation products, elements, and other analytes or sample-derived measures. Markers can include expression levels of an intracellular protein or extracellular protein. Markers can also include combinations of any one or more of the foregoing measurements, including temporal trends and differences. In some embodiments a biomarker is expression of SSTR2, or binding of a radio-labeled SSTR2 analog. An additional biomarker may be the presence of EBV, for example as assessed by the presence of Epstein-Barr Virus (EBV)-encoded small RNAs (EBER).

To “analyze” includes determining a set of values associated with a sample by measurement of a marker (such as, e.g., presence or absence of a marker or constituent expression levels) in the sample and comparing the measurement against measurement in a sample or set of samples from the same subject or other control subject(s). The markers of the present teachings can be analyzed by any of various conventional methods known in the art. To “analyze” can include performing a statistical analysis, e.g. normalization of data, determination of statistical significance, determination of statistical correlations, clustering algorithms, and the like.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group Ill: Val, lie, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

A “sample” in the context of the present teachings refers to any biological sample that is isolated from a subject. A sample can include, without limitation, an aliquot of body fluid, whole blood, tissue biopsies, synovial fluid, lymphatic fluid, ascites fluid, and interstitial or extracellular fluid. “Blood sample” can refer to whole blood or a fraction thereof. Samples can be obtained from a subject by means including but not limited to venipuncture, biopsy, needle aspirate, lavage, scraping, surgical incision, or intervention or other means known in the art.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will respond to a cancer treatment of interest.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. The term “therapeutic effect” refers to the reduction, elimination, or prevention of the disease, symptoms of the disease, or side effects of the disease in the subject.

The types of cancer that can be treated using the subject methods of the present invention include but are not limited to nasopharyngeal cancer, peripheral nervous system (PNS) cancers, sinonasal cancers, olfactory neuroblastoma, squamous cell carcinoma, adenocarcinoma, adenoid cystic carcinoma, sinonasal undifferentiated carcinoma, brain cancer, etc.

Nasopharyngeal cancer can occur in any age group, including adolescents, and is common in the South East Asia region. Although rare in the US and Western Europe, it is common in Asia and is one of the most common cancers among Chinese immigrants in the US, especially those of southern Chinese and Southeast Asian ancestry. Over several generations, the prevalence among Chinese-Americans gradually decreases to that among non-Chinese Americans, suggesting an environmental component to etiology. Dietary exposure to nitrites and salted fish also is thought to increase risk. Epstein-Barr virus is a significant risk factor, and there is hereditary predisposition.

Nasopharyngeal cancer often presents with palpable lymph node metastases in the neck. Another common presenting symptom is hearing loss, usually caused by nasal or eustachian tube obstruction leading to a middle ear effusion. Other symptoms include ear pain, purulent bloody rhinorrhea, frank epistaxis, cranial nerve palsies, and cervical lymphadenopathy. Cranial nerve palsies most often involve the 6th, 4th, and 3rd cranial nerves due to their location in the cavernous sinus, in close proximity to the foramen lacerum, which is the most common route of intracranial spread for these tumors. Because lymphatics of the nasopharynx communicate across the midline, bilateral metastases are common.

The World Health Organization (WHO) has classified nasopharyngeal cancer into 3 subtypes. Type I is classified as keratinizing squamous cell carcinoma. Type II is classified as non-keratinizing squamous cell carcinoma. Type III is classified as undifferentiated or poorly differentiated carcinoma, including lymphoepithelioma and anaplastic variants. In addition to these subtypes, nasopharyngeal cancer is also stratified based on the stage of the disease. Nasopharyngeal cancer is staged as shown below.

TABLE 1
Head and neck cancers are staged according to size and
site of the primary tumor (T), number
and size of metastases to the cervical lymph nodes (N),
and evidence of distant metastases (M).

| | Tumor (Maximum | Regional Lymph | Distant |
|---|---|---|---|
| Stage | Penetration)* | Node Metastasis† | Metastasis‡ |
| I | T1 | N0 | M0 |
| II | T1 | N1 | M0 |
| | T2 | N0, N1 | M0 |
| III | T1, T2 | N2 | M0 |
| | T3 | N0-2 | M0 |
| IVA | T4 | N0-2 | M0 |
| | Any T | N3 | M0 |
| IVB | Any T | Any N | M1 |

*T1 = tumor confined to nasopharynx or extends to oropharynx and/or nasal cavity without parapharyngeal involvement; T2 tumor extends to parapharyngeal space and/or adjacent soft tissue; T3 = tumor infiltrates bony structures at skull base, cervical vertebrae, pterygoid, and/or paranasal sinuses; T4 = tumor with intracranial extension, or involvement of cranial nerves, hypopharynx, orbit, parotid, and/or extensive soft tissue infiltration.
†N0 = none; N1 = unilateral cervical or bilateral retropharyngeal nodes ≤6 cm; N2 = bilateral cervical nodes ≤6 cm ; N3 = unilateral or bilateral node >6 cm, and/or extension below cricoid cartilage.
‡M0 = none; M1 = present.

Olfactory neuroblastoma (ONB) is an uncommon malignant neuroectodermal nasal tumor. It comprises about 2% of all sinonasal tract tumors with an incidence of approximately 0.4 per million population. ONB are thought to arise from the specialized sensory neuroepithelial (neuroectodermal) olfactory cells that are normally found in the upper part of the nasal cavity, including the superior nasal concha, the upper part of septum, the roof of nose, and the cribriform plate of ethmoid. Specifically, Jacobson's vomero-nasal organ, sphenopalatine ganglion, ectodermal olfactory placode, ganglion of Loci (nervus terminalis), autonomic ganglia of the nasal mucosa, and the olfactory neuroepithelium (cribriform plate and superomedial surface of the superior turbinate) are all sites of origination for this malignant neural crest derived neoplasm. The olfactory epithelium contains three cell types, which can be histologically identified in the tumor: basal cells, olfactory neurosensory cells, and supporting sustentacular cells. The basal cells are the stem cell compartment, continuously replacing the neurosensory cells throughout adult life, both physiologically and as a response to injury.

ONB may occur at any age, but a bimodal age distribution in the 2^{nd} and 6^{th} decades of life are most common without a gender predilection. The tumors most commonly cause unilateral nasal obstruction (70%), and epistaxis (50%), while less common signs and symptoms include headaches, pain, excessive lacrimation, rhinorrhea, anosmia, and visual disturbances. Even though the tumor arises from the olfactory neuroepithelium, anosmia is not a common complaint (5%). Due to the non-specific nature of the initial presentation and slow growth of the tumors, patients often have a long history before diagnosis. There are isolated cases reports of ONB secreting vasopressin with resultant hypertension and hyponatremia.

A “dumbbell-shaped” mass extending across the cribriform plate is one of the most characteristic imaging findings for this tumor. The observation depends on the size of the tumor and the duration of symptoms. The upper portion is a mass in the intracranial fossa, while the lower portion is in the nasal cavity, with the “waist” at the cribriform plate. CT will show speckled calcifications and bone erosion of the lamina papyracea, cribriform plate and/or the fovea ethmoidalis by non-contrast methods. Contrast enhanced CT will show homogenously enhancing mass, with non-enhancing areas suggesting regions of necrosis. ONB may rarely present with only an intracranial (frontal lobe) mass.

### Methods of Diagnosis and Treatment

As disclosed herein, a method for treatment of cancers of the head and neck, suspected of expressing somatostatin receptor 2 (SSTR2) in an individual is provided. In some embodiments the cancer is nasopharyngeal cancer or sinonasal cancers expressing SSTR2, which may be primary, local recurrent, or metastatic. In some embodiments the cancer is nasopharyngeal cancer, for example non-keratinizing World Health Organization (WHO) type II or Ill. In some embodiments the cancer is olfactory neuroblastoma.

In some embodiments, the method comprising administering an effective dose of an SSTR2 imaging agent to an individual suspected of having a tumor suspected of expressing SSTR2; and quantifying the concentration of the SSTR imaging agent within the tumor using PET-CT imaging, wherein the concentration of the imaging agent provides an indirect measurement of SSTR2 expression that can be predictive of an improved prognosis, and of responsiveness to treatment with an SSTR2 therapeutic agent. Positive expression of SSTR2 is shown to SSTR2 positivity remained predictive of an improved prognosis for patients with a hazards ratio of greater than 0.4.

In such embodiments, peptides may be labeled with a suitable radionuclide for imaging, including without limitation Gallium-68. The labeled peptide, e.g. one or more of ⁶⁸Ga-dodecane tetraacetic acid (DOTA)-(Tyr³)-octreotide or edotreotide (TOC), ⁶⁸Ga-DOTA-Nal3-octreotide (NOC), ⁶⁸Ga-DOTA-(Tyr³)-octreotate (TATE), is infused into the patient. Whole-body imaging is performed using a PET/CT system. Computed tomography (CT) may be performed with or without intravenous contrast media application for attenuation correction purposes. In some embodiments, a tumor uptake score of grade 2 or 3 is considered for therapy with an SSTR2 therapeutic agent, where the Tumor Uptake score is based on planar scinitigrams obtained 24-h post-administration of imaging and is composed of a 3-grade scale, where 1=liver uptake, 2>liver uptake and <kidney uptake and 3>kidney uptake).

In other embodiments, a biopsy sample of a cancer suspected of SSTR2 expression is contacted with an SSTR2 ligand, e.g. an anti-SSTR2 antibody or somatostatin analog, and scored based on the level of SSTR2 ligand staining, wherein the scoring of the sample provides a direct measurement of SSTR2 expression that is predictive of response to treatment with an SSTR2 agent and is in itself prognostic and predictive of clinical outcome of these diseases.

Where the methods are in vitro, the biological sample can be any sample in which a cancer cell may be present, including but not limited to, blood samples (including whole blood, serum, etc.), tissues, whole cells (e.g., intact cells), and tissue or cell extracts. Particularly, detection can be assessed on an extracellular surface of a cell. For example, the tissue sample may be fixed (e.g., by formalin treatment) and may be provided embedded in a support (e.g., in paraffin) or frozen unfixed tissue.

Assays can take a wide variety of forms, such as competition, direct reaction, or sandwich type assays. Examples of assays include Western blots; agglutination tests; enzyme-labeled and mediated immunoassays, such as enzyme-linked immunosorbent assays (ELISAs); biotin/avidin type assays; radioimmunoassays; immunoelectrophoresis; immunoprecipitation, and the like. The reactions generally include detectable labels conjugated to the affibody. Labels include those that are fluorescent, chemiluminescent, radioactive, enzymatic and/or dye molecules, or other methods for detecting the formation of a complex between antigen in the The diagnostic imaging assays described herein can be used to determine whether a subject has a cancer, as well as monitor the progress of treatment in a subject. Thus, the diagnostic assays can inform selection of therapy and treatment regimen by a clinician.

The assay reagents, including the affibodies of the present disclosure, can be provided in kits, with suitable instructions and other necessary reagents, for imaging purposes. The kit can also contain, depending on the particular assay used, suitable labels and other packaged reagents and materials (i.e. wash buffers and the like). Standard immunoassays, such as those described above, can be conducted using these kits.

In some embodiments, immunohistochemical analysis of expression of SSTR2 is performed using routine staining protocols. The slides are dichotomously scored as being positive or negative, based on the extent of staining and intensity. The extent was scored on a continuous scale from 0-100%. The intensity was scored as four categories, 0: negative (no staining); 1: weak staining not easily seen via the low power objective; 2: moderate staining still seen on a low power objective; 3: strong staining easily visible via a low power objective.

In some embodiments, immunohistochemical staining is scored with an automated analysis. An image of a stained sample deconvolved to digitally separate hematoxylin stains. Regions of tissue, white space, and artefact are manually annotated on a subset of sections, which was then used to train a tissue classifier. A deep-learning algorithm is used to detect cell nuclei and make cell level measurements. An object classifier is then trained on manually annotated areas of tumor and stroma and integrated into the pipeline to determine extent of staining in the respective regions. The output of the analysis includes the number of cell detections and H-score, where the H-score is the sum of the percentage of cells stained, multiplied by the degree of intensity, yielding scores within a range of 0 and 300. The H-score provides a measure of SSTR2 staining on the tumor, and provides prognostic classification, which can be used in determining appropriate treatment for the patient. The method may further comprise providing a computer-generated report comprising the prognosis of the condition. Expression can be graded as weak, moderate, and strong based on the intensity of staining and number of tumor cells that stain positively. An H-score can be used as a quantitative measure, where a positive tumor has an H score of at least 1.5-2 times the score of non-tumor cells in the tissue.

Disclosed herein are computer systems for implementing an automated analysis as described above, or for computing the PET analysis. A computer system includes a central processing unit (CPU, also “processor” and “computer processor” herein), which can be a single core or multi core processor, or a plurality of processors for parallel processing. The system also includes memory (e.g., random-access memory, read-only memory, flash memory), electronic storage unit (e.g., hard disk), communications interface (e.g., network adapter) for communicating with one or more other systems, and peripheral devices, such as cache, other memory, data storage and/or electronic display adapters. The memory, storage unit, interface and peripheral devices are in communication with the CPU through a communications bus, such as a motherboard. The storage unit can be a data storage unit (or data repository) for storing data. The system is operatively coupled to a computer network with the aid of the communications interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network in some cases, with the aid of the system, can implement a peer-to-peer network, which may enable devices coupled to the system to behave as a client or a server.

The system is in communication with a processing system. The processing system can be configured to implement the methods disclosed herein. In some examples, the processing system is a nucleic acid sequencing system, such as, for example, a next generation sequencing system (e.g., Illumina sequencer, Ion Torrent sequencer, Pacific Biosciences sequencer). The processing system can be in communication with the system through the network, or by direct (e.g., wired, wireless) connection. The processing system can be configured for analysis, such as nucleic acid sequence analysis.

Methods as described herein can be implemented by way of machine (or computer processor) executable code (or software) stored on an electronic storage location of the system, such as, for example, on the memory or electronic storage unit. During use, the code can be executed by the processor. In some examples, the code can be retrieved from the storage unit and stored on the memory for ready access by the processor. In some situations, the electronic storage unit can be precluded, and machine-executable instructions are stored on memory.

The program can provide a method of evaluating an H score, a tumor uptake score, etc. for clinical evaluation, and as a companion diagnostic for SSTR2 therapeutic agents.

In some embodiments, the invention provides a computer readable medium comprising a set of instructions recorded thereon to cause a computer to perform the steps of (i) receiving data from one or more nucleic acids detected in a sample; and (ii) diagnosing clonality, response to therapy, or initial diagnosis based on the quantitation.

The analysis and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and data comparisons of this invention. Such data can be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

In an individual determined to be suitable for treatment with an therapeutic SSTR2 agent, e.g. having a tumor uptake score of 2 or 3, or an H score that indicates strong expression of SSTR2, or maximized standardized uptake value (SUVmax) where a SUVₘₐₓ score of at least about 8, at least about 10, or more is considered to be a high density of somatostatin receptor, i.e. SSTR2 positive. A therapeutic SSTR2 agent can be administered by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the disclosure, by such means as depot injections or erodible implants.

In some embodiments, a peptide therapeutic SSTR2 agent is formulated at a dose of from about 0.05 mg/kg, from about 0.1 mg/kg, from about 0.5 mg/kg, from about 1 mg/kg, up to about 50 mg/kg, up to about 25 mg/kg, up to about 10 mg/kg, up to about 5 mg/kg, up to about 2.5 mg/kg, up to about 1.5 mg/kg. Radionuclide dosing may be calculated at least 0.5 Gbq/dose, at least 1 Gbq/dose, at least 2.5 Gbq/dose, at least 3 Gbq/dose, at least 5 Gbq/dose, at least 7.5 Gbq/dose, and up to about 15 Gbq/dose, and up to about 10 Gbq/dose, and up to about 7.5 Gbq/dose. Administration may be daily, every two days, every 3 days, 5 days/week, etc., for a period of time sufficient to effect treatment, e.g. 1 week, 2 weeks, 3 weeks, 4 weeks, 5 weeks, 6 weeks, 7 weeks, 8 weeks, 3 months, 4 months, or more as required.

Numerous localized drug delivery strategies have been developed to circumvent the blood brain barrier. For example, the insertion of polymeric implants that release drugs slowly into the surrounding tissue has been reported to be successful in treating tissues locally. Treatment may be enhanced by alternative delivery methods that increase the penetration distance of the drug into tissue and eliminate the rapid decay in concentration with distance that is characteristic of diffusion mediated transport. Convection-enhanced drug delivery (CED) uses direct infusion of a drug-containing liquid into tissue so that transport is dominated by convection. By increasing the rate of infusion, the convection rate can be made large compared with the elimination rate in a region of tissue about the infusion point. Thus, CED has the potential of increasing the drug penetration distance and mitigating the decay in concentration with distance from the release point.

Nanoparticles as drug or gene carriers may be used, e.g. in combination with CED. Transport of particles through the extracellular space of tissues is hindered by the large size of nanoparticles (10-100 nm), which are much larger than small molecule drugs or therapeutic proteins that more easily penetrate the brain extracellular matrix (ECM). However, nanoparticles may be able to penetrate brain tissue provided that particles are less than 100 nm in diameter, are neutral or negatively charged, and are not subject to rapid elimination mechanisms.

In some embodiments the agent is delivered as continuous intraventricular CNS administration. In some embodiments, intraventricular administration is combined with systemic administration, for example utilizing an implantable device to deliver the agent. In some embodiments the implantable device is an osmotic pump. The device may be implanted intraventricularly, for example, with a conventional stereotaxic apparatus.

A continuous delivery device includes, for example, an implanted device that releases a metered amount of an agent continuously over a period of time. The device may be implanted so as to release the anti-agent into the cerebrospinal fluid (CSF). An example of such devices is an osmotic pump, which operates because of an osmotic pressure difference between a compartment within the pump, called the salt sleeve, and the tissue environment in which the pump is implanted. The high osmolality of the salt sleeve causes water to flux into the pump through a semipermeable membrane which forms the outer surface of the pump. As the water enters the salt sleeve, it compresses the flexible reservoir, displacing the test solution from the pump at a controlled, predetermined rate. The rate of delivery is controlled by the water permeability of the pump's outer membrane. Thus, the delivery profile of the pump is independent of the drug formulation dispensed. Drugs of various molecular configurations, including ionized drugs and macromolecules, can be dispensed continuously in a variety of compatible vehicles at controlled rates.

As noted above, a therapeutic agent can be formulated with a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

Dosage and frequency may vary depending on the half-life of the agent in the patient. It will be understood by one of skill in the art that such guidelines will be adjusted for the molecular weight of the active agent, the clearance from the blood, the mode of administration, and other pharmacokinetic parameters. The dosage may also be varied for localized administration, e.g. intranasal, inhalation, etc., or for systemic administration, e.g. i.m., i.p., i.v., oral, and the like.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds useful for co-administration with the active agents of the invention can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C.). Chemicals that are known but not commercially available in catalogs may be prepared by custom chemical synthesis houses, where many of the standard chemical supply houses (e.g., those listed above) provide custom synthesis services.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

Chemotherapy may include Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (lbritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MED14736, MPDL3280A and MSB0010718C targeting PD-L1 and those targeting CTLA-4 such as ipilimumab.

The term “immune checkpoint inhibitor” refers to a molecule, compound, or composition that binds to an immune checkpoint protein and blocks its activity and/or inhibits the function of the immune regulatory cell expressing the immune checkpoint protein that it binds (e.g., Treg cells, tumor-associated macrophages, etc.). Immune checkpoint proteins may include, but are not limited to, CTLA4 (Cytotoxic T-Lymphocyte-Associated protein 4, CD152), PD1 (also known as PD-1; Programmed Death 1 receptor), PD-L1, PD-L2, LAG-3 (Lymphocyte Activation Gene-3), OX40, A2AR (Adenosine A2A receptor), B7-H3 (CD276), B7-H4 (VTCN1), BTLA (B and T Lymphocyte Attenuator, CD272), IDO (Indoleamine 2,3-dioxygenase), KIR (Killer-cell Immunoglobulin-like Receptor), TIM 3 (T-cell Immunoglobulin domain and Mucin domain 3), VISTA (V-domain Ig suppressor of T cell activation), and IL-2R (interleukin-2 receptor).

Immune checkpoint inhibitors are well known in the art and are commercially or clinically available. These include but are not limited to antibodies that inhibit immune checkpoint proteins. Illustrative examples of checkpoint inhibitors, referenced by their target immune checkpoint protein, are provided as follows. Immune checkpoint inhibitors comprising a CTLA-4 inhibitor include, but are not limited to, tremelimumab, and ipilimumab (marketed as Yervoy).

Immune checkpoint inhibitors comprising a PD-1 inhibitor include, but are not limited to, nivolumab (Opdivo), pidilizumab (CureTech), AMP-514 (MedImmune), pembrolizumab (Keytruda), AUNP 12 (peptide, Aurigene and Pierre), Cemiplimab (Libtayo). Immune checkpoint inhibitors comprising a PD-L1 inhibitor include, but are not limited to, BMS-936559/MDX-1105 (Bristol-Myers Squibb), MPDL3280A (Genentech), MED14736 (MedImmune), MSB0010718C (EMD Sereno), Atezolizumab (Tecentriq), Avelumab (Bavencio), Durvalumab (Imfinzi).

Immune checkpoint inhibitors comprising a B7-H3 inhibitor include, but are not limited to, MGA271 (Macrogenics). Immune checkpoint inhibitors comprising an LAG3 inhibitor include, but are not limited to, IMP321 (Immuntep), BMS-986016 (Bristol-Myers Squibb). Immune checkpoint inhibitors comprising a KIR inhibitor include, but are not limited to, IPH2101 (lirilumab, Bristol-Myers Squibb). Immune checkpoint inhibitors comprising an OX40 inhibitor include, but are not limited to MEDI-6469 (MedImmune).

Antibiotics, e.g. antibiotics with the classes of aminoglycosides; carbapenems; and the like; penicillins, e.g. penicillin G, penicillin V, methicillin, oxacillin, carbenicillin, nafcillin, ampicillin, etc. penicillins in combination with β-lactamase inhibitors, cephalosporins, e.g. cefaclor, cefazolin, cefuroxime, moxalactam, etc; tetracyclines; cephalosporins; quinolones; lincomycins; macrolides; sulfonamides; glycopeptides including the anti-infective antibiotics vancomycin, teicoplanin, telavancin, ramoplanin and decaplanin. Derivatives of vancomycin include, for example, oritavancin and dalbavancin (both lipoglycopeptides). Telavancin is a semi-synthetic lipoglycopeptide derivative of vancomycin (approved by FDA in 2009). Other vancomycin analogs are disclosed, for example, in WO 2015022335 A1 and Chen et al. (2003) PNAS 100(10): 5658-5663, each herein specifically incorporated by reference. Non-limiting examples of antibiotics include vancomycin, linezolid, azithromycin, daptomycin, colistin, eperezolid, fusidic acid, rifampicin, tetracyclin, fidaxomicin, clindamycin, lincomycin, rifalazil, and clarithromycin.

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

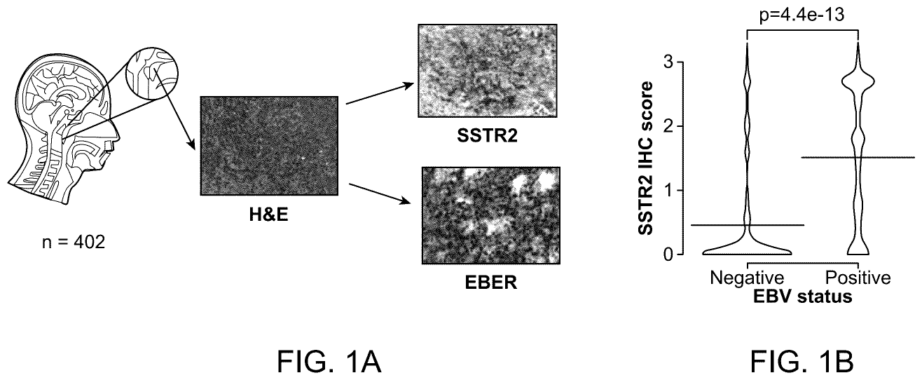

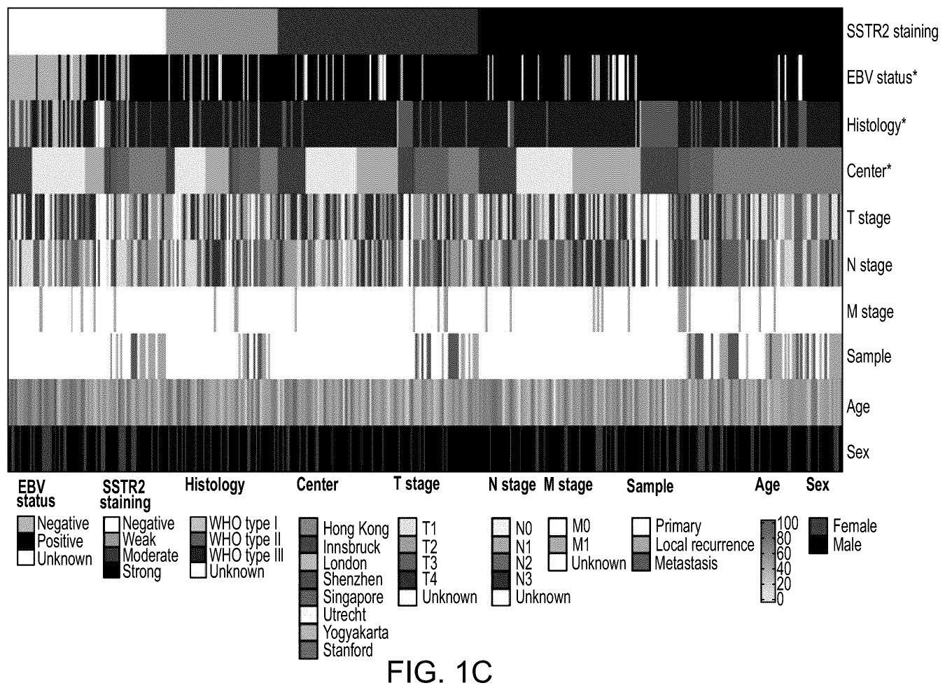

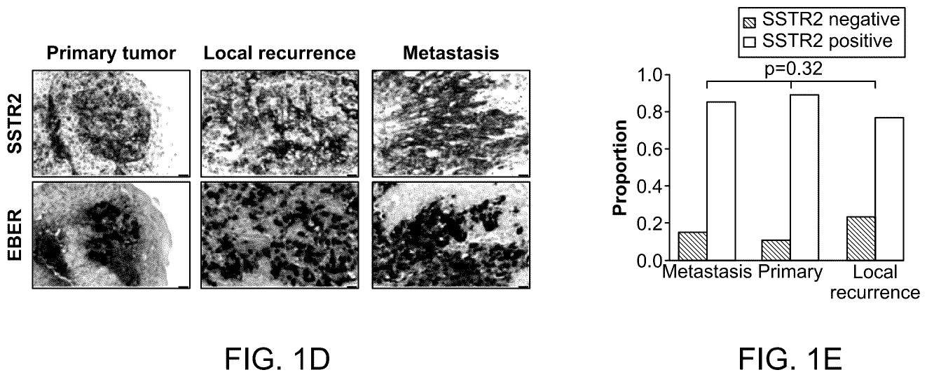

SSTR2 expression is EBV-linked in the majority of NPC. In order to validate the findings of previous studies we performed immunohistochemical staining of SSTR2 on 402 NPC primary, recurrent and metastatic tumor samples (Table 2) from European and Asian centers as well as one US center. 252 of the 311 (81%; Table 3) primary tumor samples showed SSTR2 expression, which was localized at the plasma membrane (a representative primary case is shown in FIG. 1a).

TABLE 2

| | N | % |
|---|---|---|
| Sex | | |
| Male | 285 | 70.9 |
| Female | 111 | 29.1 |
| Total | 402 | 100.0 |
| Age categorized | | |
| <65 | 325 | 80.8 |
| ≥65 | 77 | 19.2 |
| Total | 402 | 100.0 |
| Type Histology | | |
| WHO type I | 20 | 5.0 |
| WHO type II | 75 | 19.0 |
| WHO type III | 299 | 75.9 |
| Total | 394 | 100.0 |
| EBV Status | | |
| Positive | 317 | 82.3 |
| Negative | 68 | 17.7 |
| Total | 385 | 100.0 |
| Tumor Stage | | |
| T1 | 92 | 25.8 |
| T2 | 95 | 26.6 |
| T3 | 75 | 21.0 |
| T4 | 95 | 26.6 |
| Total | 357 | 100.0 |
| Nodal Stage | | |
| N0 | 76 | 21.1 |
| N1 | 99 | 27.4 |
| N2 | 129 | 35.7 |
| N3 | 57 | 15.8 |
| Total | 361 | 100.0 |
| M Stage | | |
| M0 | 296 | 91.6 |
| M1 | 27 | 8.4 |
| Total | 323 | 100.0 |
| UICC classification | | |
| Stage I | 15 | 4.7 |
| Stage II | 67 | 20.9 |
| Stage III | 108 | 33.8 |
| Stage IVA | 100 | 31.3 |
| Stage IVB | 30 | 9.4 |
| Total | 320 | 100.0 |

Characteristics of the NPC tissue samples investigated in this study. SSTR2 expression was found to be significantly-enriched in EBV-positive NPC (OR=12.7; p<0.001), in the non-keratinizing histological subtypes (OR=27.0; p<0.001), higher N stage (OR=2.3, p=0.003). No correlation was found with sex, T-stage, M-stage and overall UICC-stage.

TABLE 3
SSTR2 staining intensity

| Primary | | | Local recurrence | | Metastasis | | Total | |
|---|---|---|---|---|---|---|---|---|
| | N | % | N | % | N | % | N | % |
| Strong | 128 | 41.2 | 30 | 52.6 | 17 | 50.0 | 175 | 43.5 |
| Moderate | 80 | 25.7 | 8 | 14.0 | 9 | 26.5 | 97 | 24.1 |
| Weak | 44 | 14.1 | 6 | 10.5 | 4 | 11.8 | 54 | 13.4 |
| Negative | 59 | 19.0 | 13 | 22.8 | 4 | 11.8 | 76 | 18.9 |
| Total | 311 | 100.0 | 57 | 100.0 | 34 | 100.0 | 402 | 100.0 |

SSTR2 expression (assessed by semi-quantitative IHC scoring) of the NPC tissue samples investigated in this study.

Where data on EBV status were available (n=385 of 402 samples in total), 317/385 (82.3%) (Table 4) of the patient cohort were found to be EBV-positive and to express EBV-encoded small RNAs (EBERs) (a representative example is shown in FIG. 1a). Interestingly, SSTR2 expression was significantly enriched in EBV-positive NPC (OR=12.7; p<0.001; FIG. 1b) and in the non-keratinizing histological subtypes (OR=27.0; p<0.001) and significantly associated with other clinicopathological factors (FIG. 1c and Table 5). Furthermore, SSTR2 expression was maintained in local recurrent and metastatic disease (n=91 of 402 samples in total), with no significant difference of expression levels between cases (FIG. 1c-e).

TABLE 4

| | SSTR2 status | SSTR2 status | | |
|---|---|---|---|---|
| | (n) Positive | (n) Negative | Total | p-value |
| London | | | | |
| EBV positive | 11 | 4 | 15 | 0.52 |
| EBV negative | 1 | 1 | 2 | |
| Missing | 0 | 0 | 0 | |
| Total | 12 | 5 | 17 | |
| Innsbruck | | | | |
| EBV positive | 30 | 0 | 30 | 3.2e−7 |
| EBV negative | 5 | 11 | 16 | |
| Missing | 0 | 0 | 0 | |
| Total | 35 | 11 | 46 | |
| Utrecht | | | | |
| EBV positive | 54 | 4 | 58 | 4.9e−9 |
| EBV negative | 11 | 21 | 32 | |
| Missing | 2 | 1 | 3 | |
| Total | 67 | 26 | 93 | |
| Yogyakarta | | | | |
| EBV positive | 34 | 4 | 38 | 0.45 |
| EBV negative | 8 | 0 | 8 | |
| Missing | 10 | 0 | 10 | |
| Total | 52 | 4 | 56 | |
| Shenzhen | | | | |
| EBV positive | 26 | 2 | 28 | 0.19 |
| EBV negative | 1 | 1 | 2 | |
| Missing | 0 | 0 | 0 | |
| Total | 27 | 3 | 30 | |
| Hong Kong | | | | |
| EBV positive | 82 | 18 | 100 | NA |
| EBV negative | 0 | 0 | 0 | |
| Missing | 4 | 0 | 4 | |
| Total | 86 | 18 | 104 | |
| Singapore | | | | |
| EBV positive | 9 | 3 | 12 | NA |
| EBV negative | 0 | 0 | 0 | |
| Missing | 0 | 0 | 0 | |
| Total | 9 | 3 | 12 | |
| Stanford | | | | |
| EBV positive | 34 | 2 | 36 | 0.006 |
| EBV negative | 4 | 4 | 8 | |
| Missing | 0 | 0 | 0 | |
| Total | 38 | 6 | | |
| All centers | | | | |
| EBV positive | 280 | 37 | 317 | 3.9e−14 |
| EBV negative | 30 | 38 | 68 | |
| Missing | 16 | 1 | 17 | |
| Total | 326 | 76 | 402 | |

Stratification of SSTR2 expression and EBV status by geographic location/different sample sets (Fisher's Exact Test two-sided).

TABLE 5

| | | | Std. | | |
|---|---|---|---|---|---|
| Covariate | Class | Estimate | Error | Z | p |
| Intercept | — | −3.69 | 1.47 | −2.52 | 0.0117 |
| EBV | Negative | REF- | | | |
| | | ERENCE | | | |
| Positive | 2.34 | 0.58 | 4.03 | 5.66E−05 | |
| Histology | WHO type I | REF- | | | |
| | | ERENCE | | | |
| WHO type II | 1.11 | 0.85 | 1.32 | 0.189 | |
| WHO type III | 2.43 | 0.85 | 2.86 | 0.00422 | |
| T | T1-2 | REF- | | | |
| | | ERENCE | | | |
| T3-4 | −0.37 | 0.38 | −0.97 | 0.33 | |
| N | N0-1 | REF- | | | |
| | | ERENCE | | | |
| N2-3 | 1.07 | 0.67 | 1.6 | 0.11 | |
| M | M0 | REF- | | | |
| | | ERENCE | | | |
| M1 | 0.57 | 0.83 | 0.69 | 0.491 | |
| Center | Hong Kong | REF- | | | |
| | | ERENCE | | | |
| Innsbruck | 1.41 | 0.78 | 1.81 | 0.0708 | |
| London | −0.17 | 1.15 | −0.15 | 0.882 | |
| Shenzhen | 2.58 | 1.32 | 1.96 | 0.0505 | |
| Singapore | −1.51 | 0.92 | −1.63 | 0.102 | |
| Stanford | 2.27 | 0.86 | 2.65 | 0.00817 | |
| Utrecht | 1.03 | 0.74 | 1.39 | 0.164 | |
| Yogyakarta | 1.08 | 0.84 | 1.28 | 0.2 | |
| Sample | Primary | REF- | | | |
| | | ERENCE | | | |
| Local | −0.52 | 0.57 | −0.92 | 0.359 | |
| recurrence | | | | | |
| Metastasis | 0.3 | 0.73 | 0.41 | 0.682 | |
| Age | — | 0.01 | 0.02 | 0.65 | 0.516 |
| Sex | — | 0.47 | 0.42 | 1.13 | 0.259 |

Multivariate logistic regression model of association between clinical covariates and dichotomized SSTR2 expression (unadjusted two-sided Wald test).

TABLE 6

| Patient | | SUVmax | | | Biopsy | | IHC | |
|---|---|---|---|---|---|---|---|---|
| ID | Stage | T | N | M | Site | SUVmax | Score | Radiopeptide |
| NPC- | T4N3M1 | 18.9 | 16.1 | 15.1 | PNS | 18.9 | 3 | DOTA-TATE |
| 002 | | | | | | | | |
| NPC- | T3N3M1 | 10.4 | 12.5 | 7.2 | PNS | 10.4 | 3 | DOTA-TATE |
| 007 | | | | | | | | |
| NPC- | T2N2M1 | 8.7 | 9.9 | 1.1 | PNS | 8.7 | 2 | DOTA-TATE |
| 008 | | | | | | | | |
| NPC- | T3N2M1 | 4.9 | 7.8 | 14.9 | PNS | 4.9 | 3 | DOTA-TATE |
| 013 | | | | | | | | |
| NPC- | T2N2M1 | 6.4 | 7.3 | 12.3 | PNS | 6.4 | 3 | DOTA-TATE |
| 0′7 | | | | | | | | |
| NPC- | rT3N0M0 | 12.2 | — | — | PNS | 12.2 | 3 | DOTA-TATE |
| 019 | | | | | | | | |
| NPC- | rT0N0M1 | — | 4.1 | 1.5 | Lung | 1.5 | 0 | DOTA-TATE |
| 005 | | | | | | | | |
| NPC- | T3N3M0 | 4.9 | 3.4 | — | PNS | 4.9 | 1 | DOTA-TATE |
| 006 | | | | | | | | |
| NPC- | T1N2M0 | 5.4 | 10.0 | — | PNS | 5.4 | 0 | DOTA-TATE |
| 012 | | | | | | | | |
| NPC- | T3N3M1 | 4.3 | 22.1 | 13.8 | Skin | 1.4 | 1 | DOTA-TATE |
| 015 | | | | | | | | |
| NPC- | T3N3M1 | 8.1 | 6.5 | 3.7 | PNS | 8.1 | 1 | DOTA-TATE |
| 018 | | | | | | | | |
| NPC- | T1N3M1 | 4.0 | 11.1 | 12 | PNS | 4.0 | 1 | DOTA-TATE |
| 020 | | | | | | | | |
| NPC- | T4N2M1 | 13.4 | 11.3 | 15.3 | — | — | — | DOTA-TATE |
| 003 | | | | | | | | |
| NPC- | rT4N1M1 | 2.8 | 3.2 | 7.4 | — | — | — | DOTA-TATE |
| 009 | | | | | | | | |
| NPC- | T4N3M1 | 9.4 | 4.8 | 10.4 | — | — | — | DOTA-TATE |
| 014 | | | | | | | | |

Clinical characteristics and SSTR2 status of NPC patients undergoing ⁶⁸DOA-peptide PET-CT imaging.

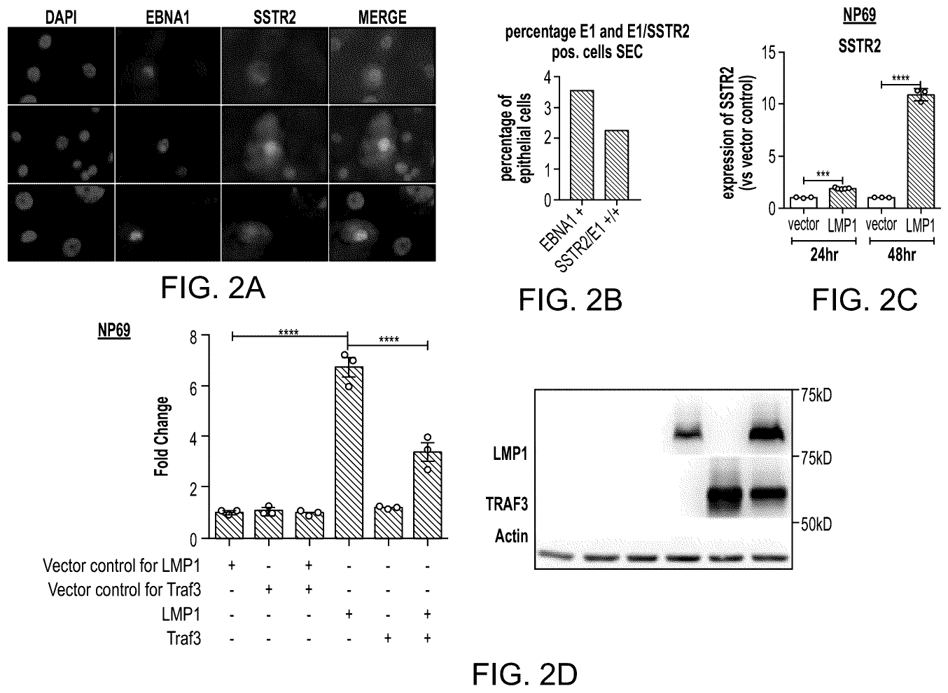

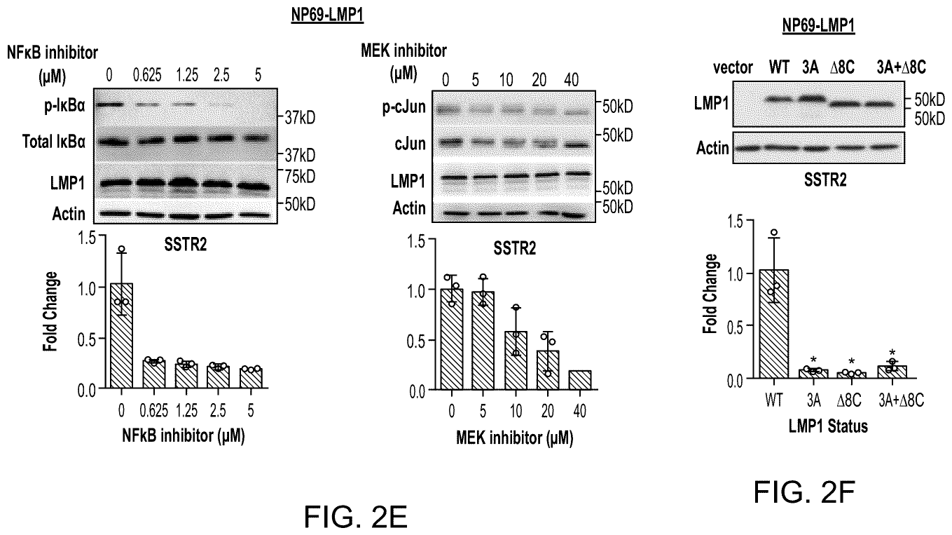

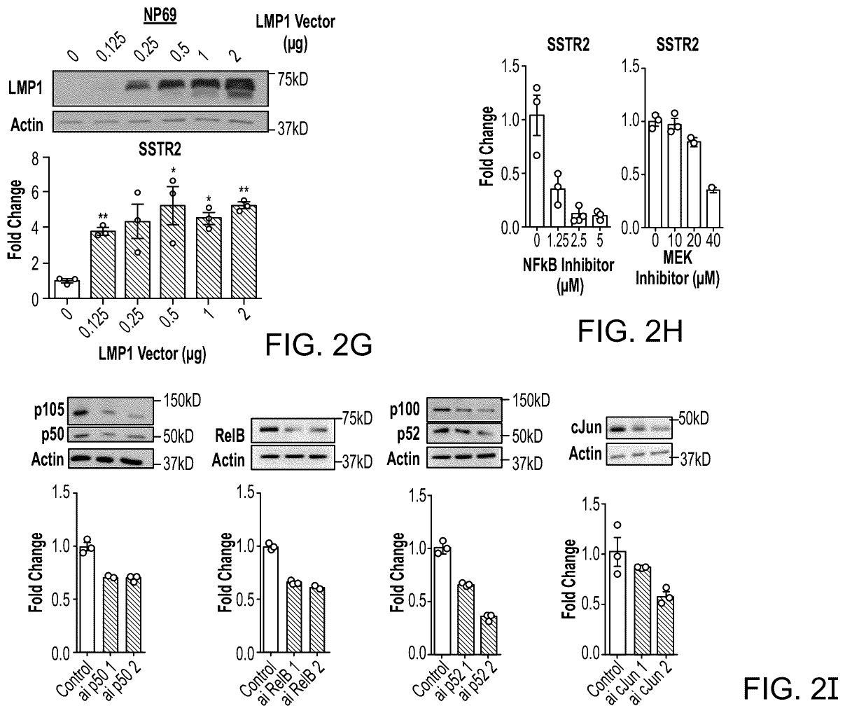

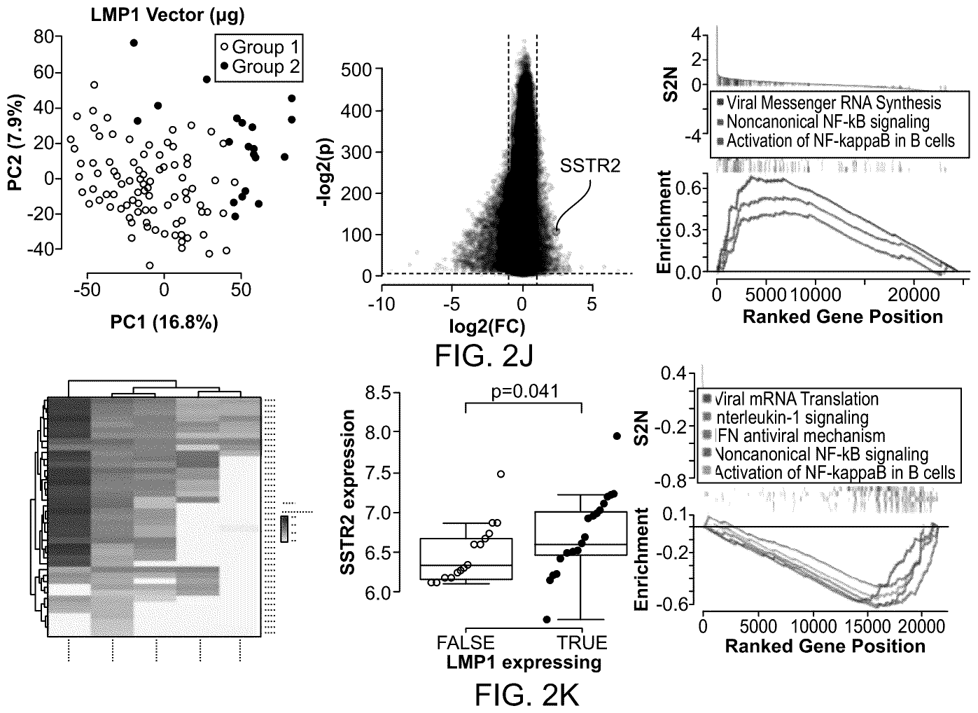

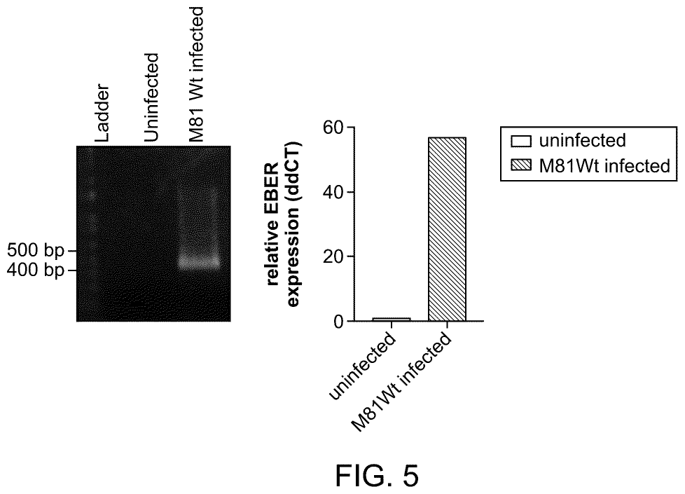

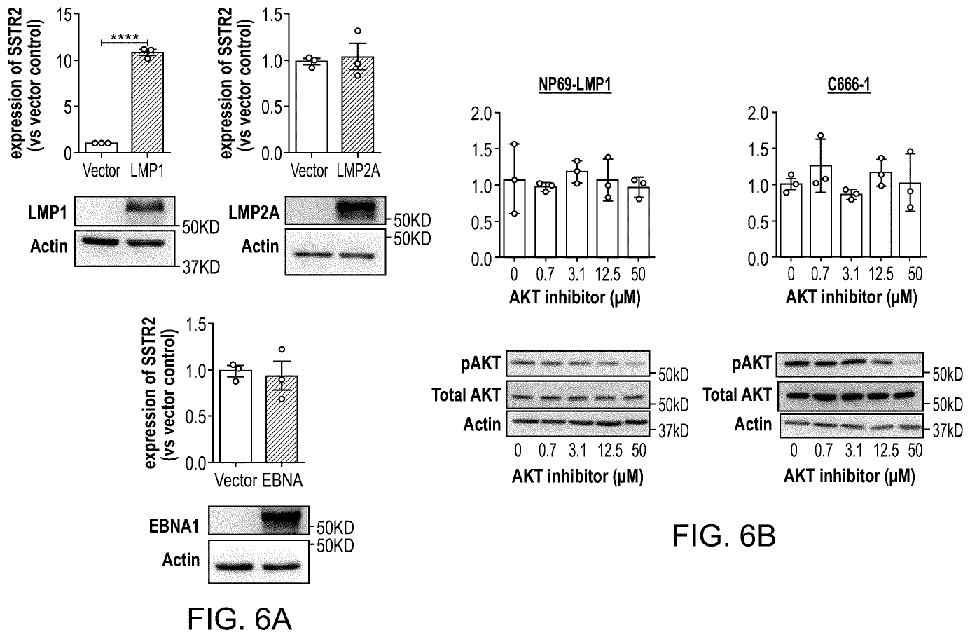

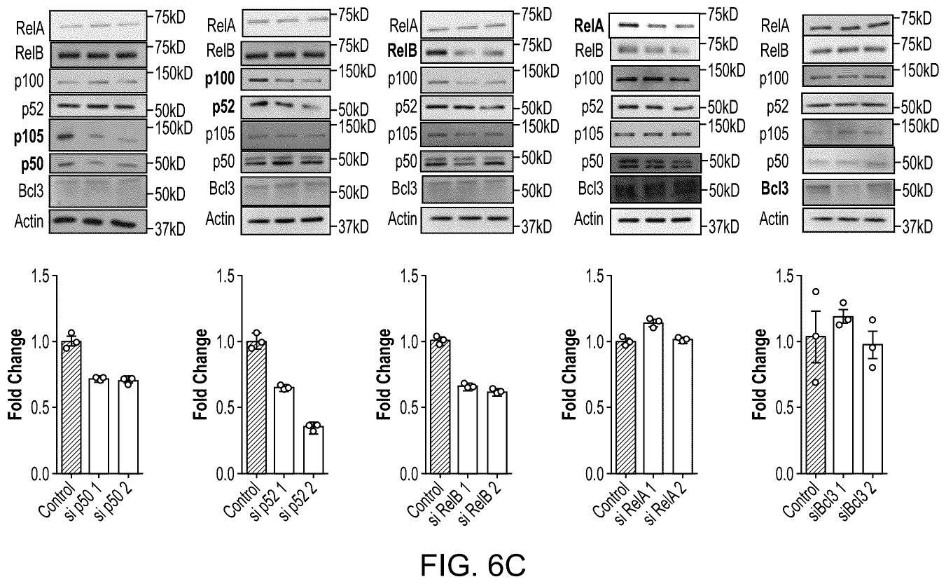

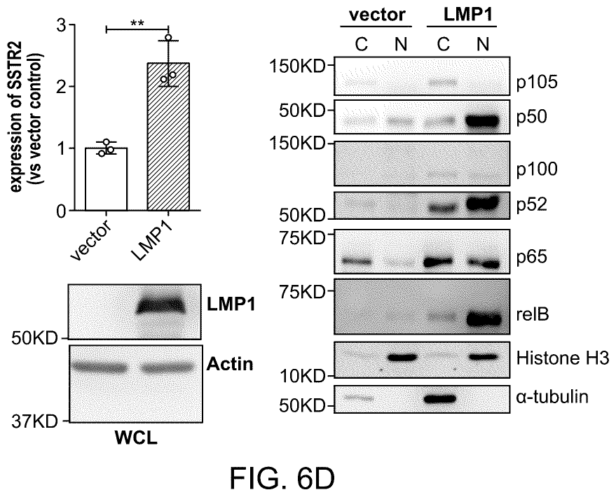

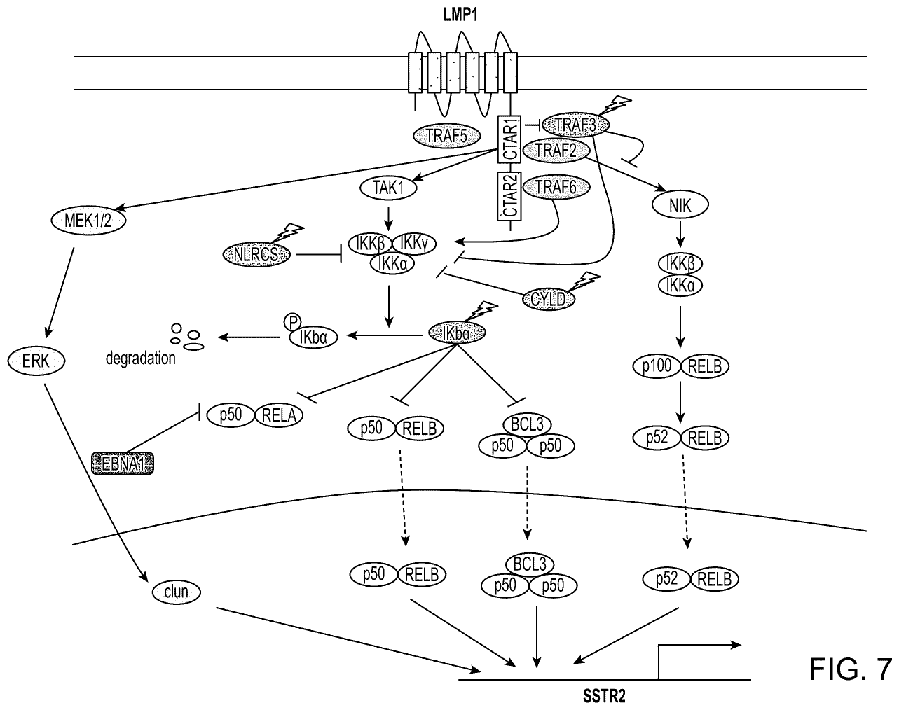

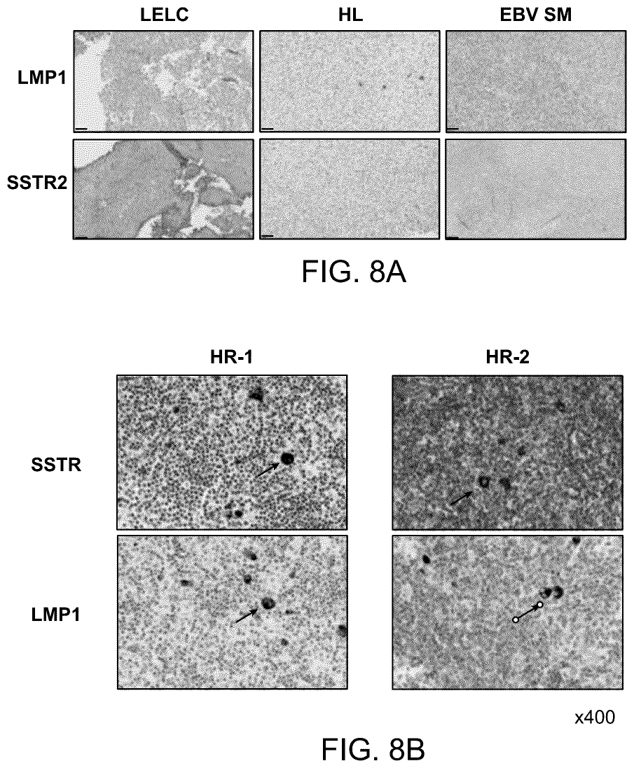

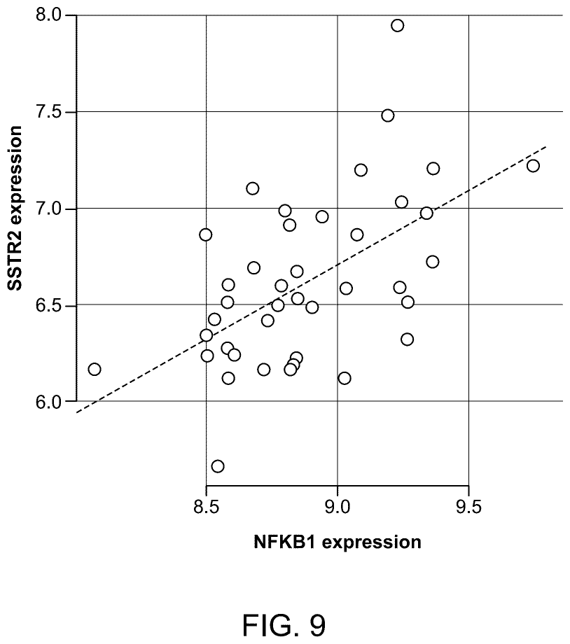

EBV induces SSTR2 expression via LMP1 and NF-κB signaling. We explored a possible relationship of EBV infection and SSTR2 expression. EBV infection of cultured primary cells from normal respiratory epithelium led to a significant upregulation of SSTR2 expression (FIG. 2a, b and FIG. 5). Aberrant activation of NF-κB signaling, either through expression of the LMP1 (latent membrane protein 1) oncoprotein of EBV or somatic mutation of negative regulators of NF-κB (e.g., in TRAF3, CYLD, NFκBIA, NLRC5) has been shown to play a driver role in NPC tumorigenesis. Transient expression of LMP1 into the immortalized nasopharyngeal epithelial cell line NP69 induced SSTR2 transcription (FIG. 2c, FIG. 6a) indicating that EBV potentially upregulates SSTR2 expression through NF-κB signaling. This SSTR2 expression was suppressed by co-expression of TRAF3, a negative regulator of NF-κB (FIG. 2d) or by treatment with the NF-κB inhibitor BAY 11-7085 (FIG. 2e). Moreover, LMP1 proteins with mutant CTAR1 and CTAR2 domains, known to be critical for activation of NF-κB by LMP1 (FIG. 6), induced significant lower SSTR2 expression compared with NPC transfected with wild-type LMP1 (FIG. 2f, g). In addition to contributing to NF-κB signaling, the CTAR1 domain of LMP1 also leads to activation of the AKT and MEK/ERK pathways. Using pharmacological inhibitors of these pathways indicated that LMP1-activated SSTR2 expression induced signaling by MEK but not AKT (FIG. 2e, FIG. 6b). As a complementary approach to LMP1 transfection in NP69 cells, we used the C666-1 NPC cell line in which NF-κB signaling is known to be endogenously-activated via somatic mutation of its negative regulators TRAF3, CYLD, and TNFAIP3. SSTR2 expression in C666-1 cells was suppressed by the NF-κB inhibitor BAY 11-7085 and the MEK inhibitor U0126 (FIG. 2h) or upon siRNA-driven downregulation of subunits of activated NF-κB signal complexes (NFκB1 (p105/p50), NFκB2 (p100/p52) or RELB) and c-Jun (FIG. 2i, FIG. 6c, d). Taken together, these findings indicate that EBV infection induces SSTR2 expression in nasopharyngeal epithelial cells through expression of the latent oncoprotein LMP1 and activation of the NF-κB and MEK signaling pathways (FIG. 7). This observation was confirmed further in other EBV-induced cancers (FIG. 8).

EBV-driven signaling pathways involved in SSTR2 expression. To validate our finding of an association between SSTR2 expression and EBV infection, we analyzed an independent NPC cohort where gene expression data were available for 113 samples. Unsupervised hierarchical clustering and principal component analysis (PCA) based on gene expression revealed two groups of samples in this dataset (FIG. 2j, left panel). Differential gene expression analysis between these groups revealed overexpression of SSTR2 in group 1 tumors (FIG. 2j, middle panel). Furthermore, geneset enrichment analysis (GSEA) revealed an upregulation of pathways related to viral infection in group 1 samples compared to the other group (FIG. 2j, right panel). Further, a supervised analysis on another dataset where both EBV gene expression (FIG. 2k, left panel) and microarray human gene expression were available, revealed a positive correlation between viral LMP1 expression and tumor SSTR2 expression (FIG. 2k, middle panel), tumor NFκB1 expression and SSTR2 expression (FIG. 8) and upregulation of viral biogenesis pathways in LMP1-expressing samples compared to non-LMP1-expressing samples (FIG. 2k, right panel).

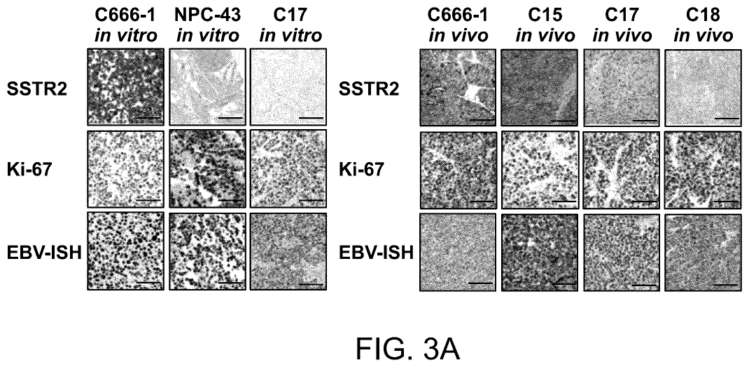

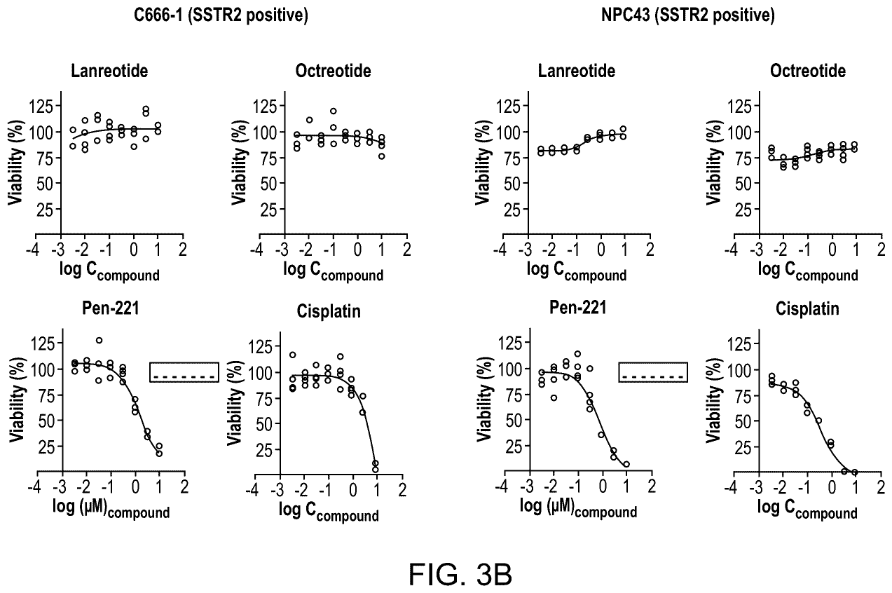

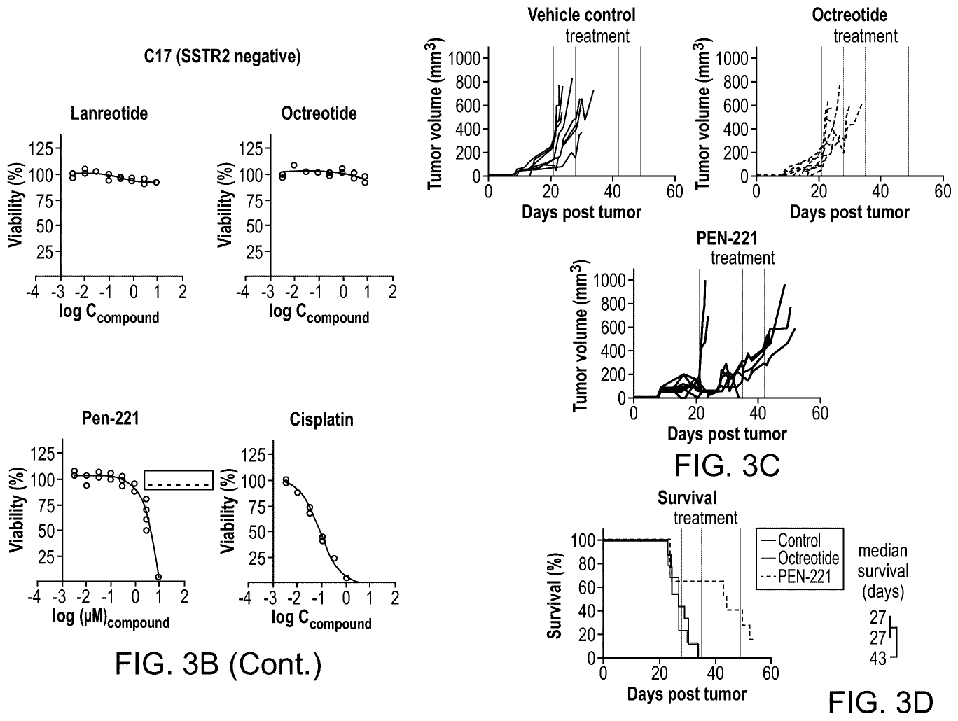

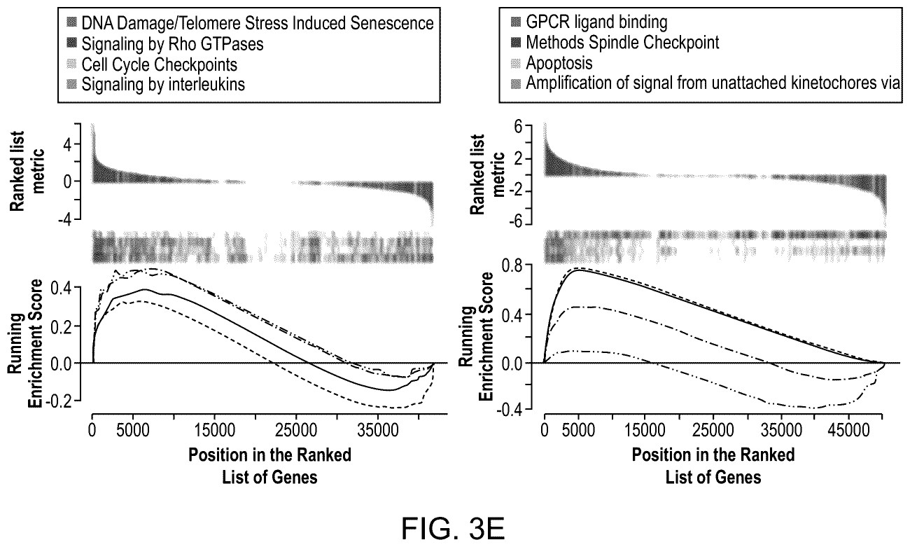

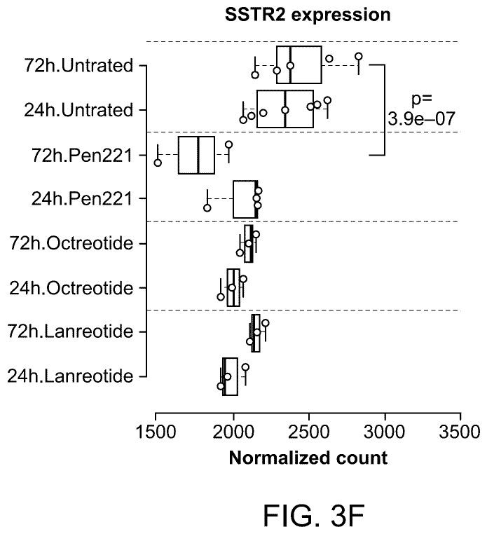

Antitumor effect of PEN-221, an anti-SSTR2 drug conjugate. We then hypothesized that SSTR2 expression observed in patients sensitizes NPC to SSTR2-targeted cytostatic or cytotoxic agents. To investigate this, we used the well-characterized EBV positive NPC cell lines C666-1, NPC43, and C17. When taken from in vitro cultures or in vivo xenografts, C666-1 and NPC43 showed high expression of EBERs, SSTR2 and the Ki-67 proliferation antigen whereas C17 was found to be SSTR2 negative (FIG. 3a). We next tested the impact on in vitro proliferation/survival of these cell lines of cisplatin, a chemotherapeutic agent used in the treatment of patients with NPC, and a range of SSTR2 agonists, including the FDA-approved lanreotide and octreotide and PEN-221, which is in Phase 1/2a clinical trial in the UK and US for NET patients. PEN-221 is a drug conjugate made up of a peptide that is highly selective for SSTR2 conjugated to the tubulin polymerization inhibitor DM1 and has shown effectiveness in preclinical models. Unlike in NETs, lanreotide and octreotide did not affect in vitro proliferation of C666-1 and NPC43, in contrast to PEN-221 (FIG. 3b and FIG. 7). We next subcutaneously xenografted C666-1 cells into athymic nude mice and treated established tumors with octreotide (n=9), PEN-221 (n=8), or vehicle control (n=9). Mice treated with PEN-221 showed a significant increase in overall survival (p=0.0368; Log-rank Mantel-Cox test) (FIG. 3c, d), further indicating superior anti-tumor efficacy of PEN-221 as compared to octreotide.

Molecular effects of in vitro cytotoxic payload of PEN-221. We next performed RNA-seq to explore the molecular effects of SSTR2-targeted drugs on C666-1 cells. Lanreotide and octreotide did not affect SSTR2 expression, but induced upregulation of pathways related to somatostatin biology such as interleukin signaling as well as upregulation of cell senescence pathways 24 h post-treatment (FIG. 3e shows data for lanreotide), but no changes in cell death/apoptotic pathways. In contrast, 72-h treatment with PEN-221 led to significant downregulation of SSTR2 expression (FIG. 3f) as well as upregulation of pathways related to apoptotic signaling and mitotic spindle formation dysregulation, the latter in keeping with the mechanism of the cytotoxic payload of PEN-221.

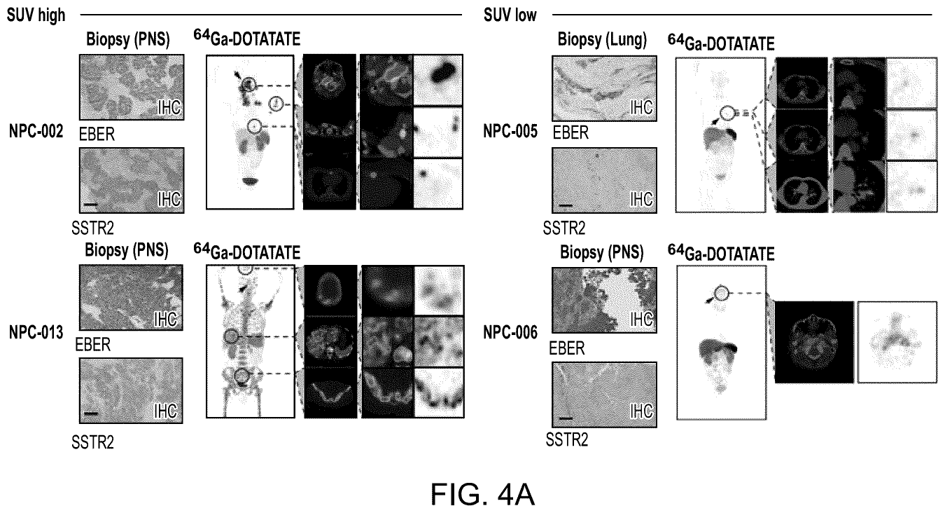

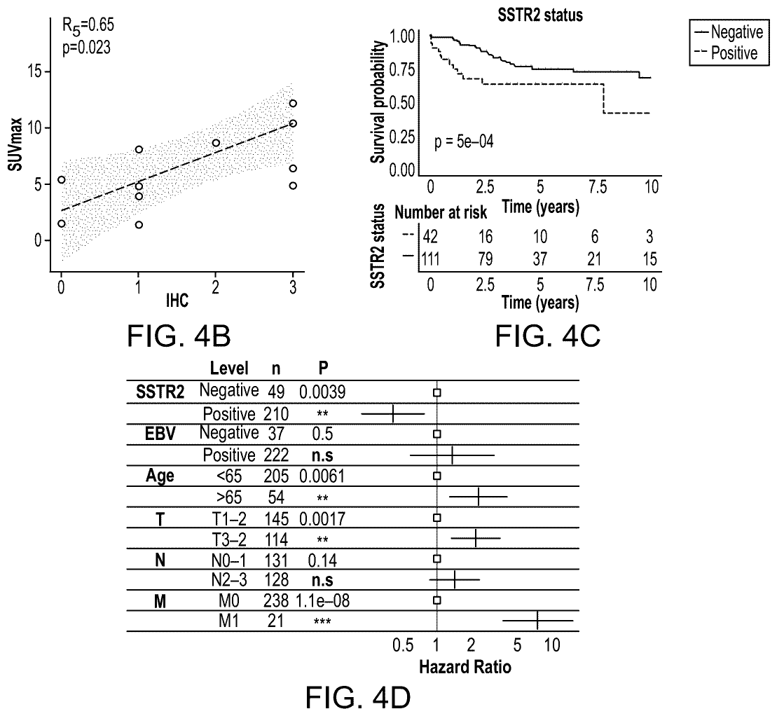

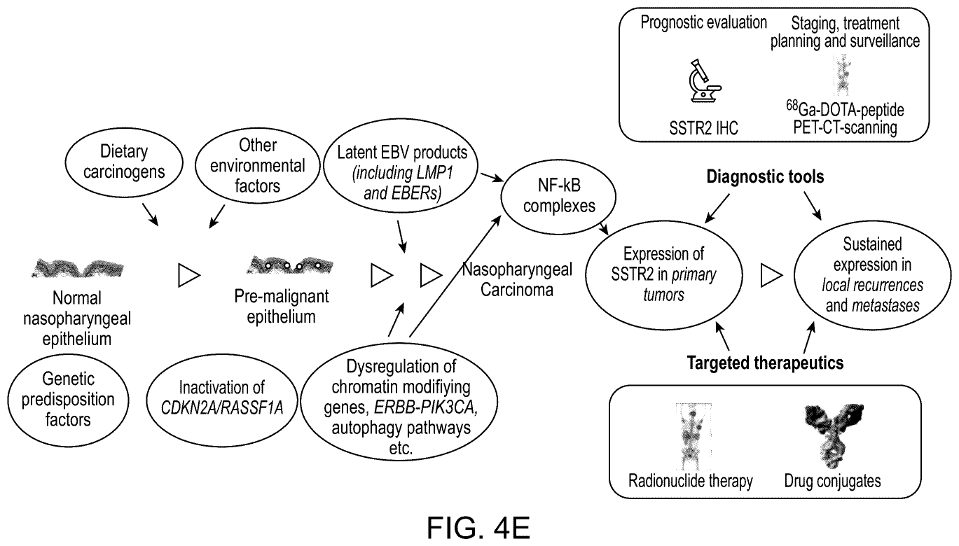

SSTR2 as a diagnostic NPC biomarker in the clinic. In a clinical trial of NPC patients (NCT03670342) we show the use of SSTR2 as a noninvasive biomarker in NPC and integrated SSTR2 protein expression data with ⁶⁸Ga-DOTA-peptide imaging data on 12 patients. We found a significant correlation of SSTR2 expression levels with in vivo uptake of ⁶⁸Ga-DOTApeptides (FIG. 4a, b), indicating the utility of this imaging modality as a noninvasive marker to monitor SSTR2 expression and as a target for SSTR2 receptor-targeted radionuclide therapy (Lutetium-177, Ytrium-90).

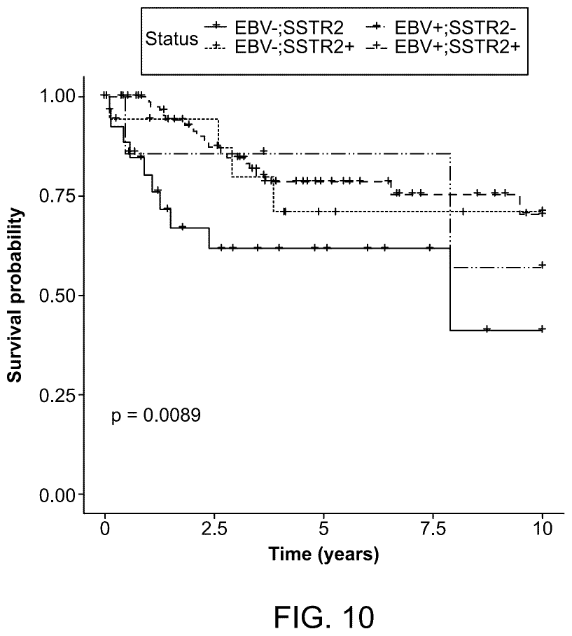

SSTR2 expression is a prognostic biomarker in NPC. Analysis of overall survival of the study population in patients from the European centers (where standards of treatment were comparable) revealed improved survival in NPC patients with tumors positive for SSTR2 (p<0.001) (FIG. 4c). Jointly classifying patients by EBV and SSTR2 status revealed the poorest prognosis for those patients who are both EBV negative and SSTR2 negative (FIG. 10). Further, in a multivariate cox regression analysis with SSTR2 status, EBV status, patient age, T, N, and M staging in all patients for which such information was available (n=209), SSTR2 positivity remained predictive of an improved prognosis for patients (Hazard ratio (HR)=0.41, where hazard rate ratio=treatment hazard rate/placebo hazard rate (FIG. 4d). Importantly, in this analysis, EBV status was not prognostic independently of SSTR2 status.

### Methods

Materials. Four hundred and two formalin-fixed paraffin-embedded (FFPE) NPC specimens were obtained from three European institutions (University College London/University College London Hospital, UK; Medical University of Innsbruck, Austria; University Medical Center in Utrecht/UMCU, Netherlands), one US institution (Stanford University, Palo Alto, US) and from four institutions in Asia (Gadjah Mada University/Dr. Sardjito Hospital, Yogyakarta, Indonesia, The Chinese University of Hong Kong, Hong Kong SAR, Jinan University, Shenzhen, Guangdong, China, and National Cancer Centre Singapore). Ethical approval was obtained from all institutions (Yogyakarta, Indonesia: KE/FK/0198/EC/2017; Singapore: 2015/2482; Hong Kong, China: CREC-2013-229; Shenzhen, China: LL-KY-2019143; London, UK: UCL 04/0099; REC 04/Q0505/59; Stanford, US: IRB-43567; Innsbruck, Austria: AN2014-0241, 340/4.20; Utrecht, Netherlands: TCBio 14/510) with further ethical approval for multicenter data analysis from University College London Research Ethics Committee (UCL REC no. 9609/002). Histological characterization and sample selection/cohort was performed by two head and neck pathologists (SWI; SS) both experienced in the evaluation of NPCs. The date of diagnosis was defined as the date of tissue extraction for histological determination of the diagnosis. The end date for OS was the date of death.

Tissue microarray construction. Tissue microarrays (TMA) were constructed from the 93 specimens from University Medical Center in Utrecht/UMCU, Netherlands. The TMAs were constructed with a TMA Grand Master instrument (3D HISTECH, Budapest, Hungary) using the respective FFPE blocks. Tumor areas were marked by a pathologist (SWI) and pathology resident (MOO) experienced in the histological evaluation of NPCs. Three cores (0.6 mm) were punched from the marked tumor areas and arrayed into a recipient TMA donor block.

Immunohistochemical analysis of expression of SSTR2, Ki-67 Chromogranin A and Synaptophysin. Immunohistochemistry was performed in different institutions, almost all using Ventana automated staining instruments (Ventana Medical systems, Tucson, AZ, USA). The Singapore team used a Leica Bond-Max (Leica Biosystems, Wetzlar, Germanny) autostainer for these purposes. For detection of chromogranin A (LK2H10; Ventana), synaptophysin (SP11; Ventana) and Ki-67 (Clone MIB-1, DAKO, Glostrup, Denmark), routinely available staining protocols were used. For detection of SSTR2, the rabbit monoclonal antibody UMB1 (Abcam, Cambridge, UK) was used. In Utrecht an anti-SSTR2 antibody (rabbit polyclonal, code SS-8000-RM, diluted 1:5, BioTrend, Cologne, Germany) was used during the initial run and further validated with the above antibody (FIG. 10). The slides were evaluated under the guidance of head and neck pathologists (SWI, SS). The evaluators of the immunohistochemical stains were blinded to the clinical outcomes or EBV status and membranous staining of the tumor cells was assessed (FIG. 1). The slides were dichotomously scored as being positive or negative, based on the extent of staining and intensity. The extent was scored on a continuous scale from 0-100%. The intensity was scored as three categories (1: weak staining not easily seen via the low power objective; 2: moderate staining still seen on a low power objective; 3: strong staining easily visible via a low power objective).

EBV status. EBV status was determined by Epstein-Barr virus-encoded Early RNA (EBER) in situ hybridization (ISH) on the samples, brushes or TMA. Ventana BenchMark automated staining instruments (Ventana Medical systems, Tucson, AZ, USA) were used for ISH of the samples or TMA using an EBV-specific probe (INFORM EBER PROBE; Ventana Medical systems) and ISH iVIEW Blue detection kit (Ventana Medical systems, Inc.) for staining using the manufacturer's instructions in Innsbruck, Utrecht, Hong Kong, Stanford and London. Shenzhen used an EBER Probe (Zhongshan Jinquaiao Biotechnology Co.; Beijing, China) and an autostainer (Ventana Medical Systems, Inc.) to perform ISH. Singapore used a BOND™ Ready-to-use ISH EBER probe and a Leica Bond-Max autostainer (all Leica Biosystems, Wetzlar, Germany) for this purpose. In situ hybridization of xenografts and cell pellets was done using an EBV-specific probe (INFORM EBER PROBE; Ventana Medical systems) and ISH iVIEW Blue detection kit (Ventana Medical systems, Inc.) using the manufacturer's instructions.

EBV infection of primary epithelial cells. Primary epithelial cells were grown on glass slides in serum-free keratinocyte growth medium (KGM-SFM, Thermo Fisher Scientific, US) at 37° C., 5% CO₂. Cells were exposed to 5×10⁷ M81 viruses for 72 h, followed by virus removal and washing of the cells with 1×PBS. The slides were dried, fixed with 4% PFA for 15 min, permeabilized for 10 min in 1×PBS/0.1% Triton-X100. After a 30 min blocking step and cells being kept in in 1% BSA/PBS at 37° C. for 4 min, the slides were incubated with a rabbit antibody directed against human SSTR2 (clone 11 HCLC, Thermo Fisher; dilution 1:200 in 1% BSA/PBS) for 12 h at 4° C. The slides were then washed and incubated with a rat antibody specific for EBNA1 (clone 1H4, provided by R. Feederle, Munich, Germany; dilution 1:10 in 1% BSA/PBS) for 2 h at 37° C. in a humidified chamber. After three washes steps, the cells were incubated with a secondary goat anti-rabbit antibody coupled to Alexa 488 (A11008, Invitrogen; dilution 1:300) and with a secondary goat anti-rat antibody coupled to Cy3 (112-165-143, Dianova; dilution 1:900) for 30 min at 37° C. Cell nuclei were counterstained with DAPI (40 ng/ml). Slides were analyzed using a Leica epifluorescence microscope equipped with a CCD camera. qPCR: After DNAse treatment, Trizol-purified RNA was reverse transcribed with AMVreverse transcriptase (Roche) using a mix of random primers. EBER transcripts were detected by quantitative PCR using specific primers (EBER1 fwd 5′-acgctgccctagaggttttg-3′, EBER1 rev 5′-gcagaaagcagagtctggga-3′) and probes (EBER1 probe 5′FAM-aggacggtgtctgtggttgt-3′TAMRA) for 40 cycles using the universal thermal cycling protocol on an ABI STEP ONE PLUS Sequence Detection System (Applied Biosystems). All RT-PCRs included samples not treated with reverse transcriptase that served as negative controls. All samples were run in duplicate, together with primers specific for the human GAPDH gene to normalize for variations in cDNA recovery. SSTR2 transcripts were amplified using specific primers (SSTR2 fwd GAAGAGAATCAATAGCGTGTTTTATTGCATGTC, SSTR2 rev CATAGCGGAGGATGACATAAATGAC) for 40 cycles. Non-infected primary epithelial cells served as a negative control. All used primers are listed in Table 7.

TABLE 7

| Appli- | | |
|---|---|---|
| cation | Name | Sequence |
| PCR | SSTR2 | F: CTTTCTTGGCTATGCAGGTGG |
| | | (SEQ ID NO: 1) |
| | | R: GAAGATGCTGGTGAACTGATTG |
| | | (SEQ ID NO: 2) |
| | SSTR2 | F: GCACAAGAGGGTCGAGGAG |
| | | (SEQ ID NO: 3) |
| | | R: CATAGCGGAGGATGACATAAATGAC |
| | | (SEQ ID NO: 4) |
| | EBER1 | F: ACGCTGCCCTAGAGGTTTTG |
| | | (SEQ ID NO: 5) |
| | | R: GCAGAAAGCAGAGTCTGGGA |
| | | (SEQ ID NO: 6) |
| | EBER1 | Fam-AGGACGGTGTCTGTGGTTGT-Tamra |
| | probe | (SEQ ID NO: 7) |
| SiRNA | NFKB1 | siRNA 1: |
| | | AUAUUUGAAGGUAUGGGCCAUCUGC |
| | | (SEQ ID NO: 8) |
| | | siRNA 2: |
| | | UUAUACACGCCUCUGUCAUUCGUGC |
| | | (SEQ ID NO: 9) |
| | RelB | siRNA 1: |
| | | GAGGACAUAUCAGUGGUGUUCAGCA |
| | | (SEQ ID NO: 10) |
| | | siRNA 2: |
| | | GCGAGGAGCUCUACUUGCUCUGCGA |
| | | (SEQ ID NO: 11) |
| | p52 | siRNA 1: |
| | | CCCAGGUCUGGAUGGUAUUAUUGAA |
| | | (SEQ ID NO: 12) |
| | | siRNA 2: |
| | | GAUUUCAAAUUGAACUCCUCCAUUG |
| | | (SEQ ID NO: 13) |
| | c-Jun | siRNA 1: |
| | | GAUGGAAACGACCUUCUAU |
| | | (SEQ ID NO: 14) |
| | | siRNA 2: |
| | | GUCAUGAACCACGUUAACA |
| | | (SEQ ID NO: 15) |
| | RelA | siRNA 1: |
| | | CCCUUUACGUCAUCCCUGA |
| | | (SEQ ID NO: 16) |
| | | siRNA 2: |
| | | GGAGUACCCUGAGGCUAUA |
| | | (SEQ ID NO: 17) |
| | Bcl3 | siRNA 1: |
| | | UACAUUUGCGCGUUCACGUUGGCGC |
| | | (SEQ ID NO: 18) |
| | | siRNA 2: |
| | | AGCUGCACCAUGCUAAGGCUGUUGU |
| | | (SEQ ID NO: 19) |

Culture of the cell lines. C666-1 cells were cultured in RPMI-1640 with 25 mM Hepes (Lonza, Berlin, Germany) supplemented with 10% heat-inactivated FCS (Gibco, Carlsbad, CA), 2 mM L-Glutamine, 100 units/ml penicillin (Gibco), and 0.1 mg/ml streptomycin (Gibco). Cells were maintained at 37° C., 5% CO₂ and passaged every 7 days at a 1:2 ratio using accutase (Sigma, St. Louis, MO, USA). Seventy-five percent of culture medium was replaced by fresh medium every 2-3 days. NPC43 and C17 cell lines were cultured in RPMI-1640 with 25 mM Hepes (Lonza, Berlin, Germany) supplemented with 10% heat-inactivated FCS (Gibco, Carlsbad, CA), 2 mM L-Glutamine, 100 units/ml penicillin (Gibco) and 0.1 mg/ml streptomycin (Gibco) and 4 μM Y27632 (Promocell, Heidelberg, Germany). Cells were maintained at 37° C., 5% CO₂ and passaged 5 days at a 1:4 ratio using accutase (Sigma, St. Louis, MO, USA). Culture medium was replaced by fresh medium every 2 to 3 days.

Immunohistochemical analysis of SSTR2 in cell pellets. Routinely cultured cell lines (2-4×10⁶) were collected by centrifugation and embedded as cell pellet in agarose as published before: Cells were harvested by centrifugation at 290×g for 10 min at 4° C., and the resulting pellet was fixed in 10 ml neutral-buffered 4% formaldehyde solution (Flintsbach am Inn, Germany). After fixation the cells were centrifuged by 400×g for 10 min at room temperature. The cell pellet was resuspended in 300 μl PBS, transferred to Eppendorf tube (1.5 ml), and kept on ice. Low melting point agarose (with gelling temperature point 34-37° C.) was prepared in PBS as 3% solution in labor glassware by microwave warming and equilibrated in a thermoblock to 65° C. for at least 30 min. The 300 μl PBS-cell suspension was also equilibrated to 65° C. for not more than 10 min. 600 μl melted eqilibrated agarose was pipetted to the cell suspension, followed by spinning at 2000×g for 5 min at room temperature. After that, the tube was placed on ice, the cell pellet was trimmed and was placed in embedding cassette. The cell pellet in the cassette was stored in PBS containing 0.05-0.1% sodium azide until embedded in paraffin in a Histos (Histocom, Wr. Neudorf, Austria) paraffin embedding system, following the instructions of the manufacturer. After embedding, biopsies were sectioned and used for in situ hybridization and immunohistochemistry. Embedded specimens were serially sectioned at 5 μm thickness using a HM 355 S microtome (Microm, Walldorf, Germany) and affixed onto Superfrost™ Plus slides (Menzel, Braunschweig, Germany). The mounted specimens were then dried overnight at room temperature, following which the slides were incubated at 60° C. for 1 h to enable the sectioned specimens to adhere firmly onto the glass surface. Immunohistochemical analysis of SSTR2 expression was performed as above.

Drugs. Lanreotide and octreotide acetate were obtained from Abcam® (Cambridge, UK). Belinostat (PXD101) was obtained from Selleckchem (Houston, US), Cisplatin ‘Ebewe’ from Sandoz (Holzkirchen, Germany) and PEN-221 was provided by Tarveda Therapeutics (Watertown, Massachusetts, US).

MTT-based cell viability assay. Two days prior addition of test compounds, NPC cells were plated at a density of 2×10⁴ cells/well in 96-well plates. Compounds were prepared as advised by manufacturers, diluted half logarithmic in medium and added to the wells in quadruplicate starting from 10 μM (30 μM for cisplatin). PEN-211 containing medium was removed after 2 h, cells were washed with PBS and fresh medium was added. Plates were incubated in a humified chamber for 7 days; 50% of medium were exchanged every other day with fresh drug dilutions. MTT (3-[4,5-dimethylthiazol-2-yl]-2,5 diphenyl tetrazolium bromide) reagent (20 μl/well of 5 mg/ml solution) (Sigma-Aldrich) was added and solubilized after 4 h using 100 μl of a 0.1 mg/ml SDS/0.01M HCl solution. Dual absorbance was measured after 5 h in a microplate reader (Epoch BioTek, BioTek Instruments, Bad Friedrichshall, Germany) using 550 nm as measurement and 655 nm as reference filter. After subtraction of background absorbance, fractions of surviving cells were obtained by normalization to the mean of nontreated samples.

RNA-seq analysis. C666-1 cells were seeded at a density of 1×10⁶/well in six-well plates 2 days prior to compound addition. PEN-221 was prepared as advised by the manufacturer and 5 μM were added in triplicates. Untreated and DMSO-treated (1:2000 in medium) wells were used as controls. 24 and 72 h post-PEN-221 addition, DNA and RNA extraction was performed from separate wells using Quick-DNA Plus and Direct-zol RNA Plus Kit (Zymo Research, Irvine, CA, USA) according to the manufacturers' instructions.

RNA concentration was normalized to 100 ng/50 μl and Illumina libraries prepared using a NEBNext Poly(A) mRNA Magnetic Isolation Module in conjunction with a NEBNext Ultra II Directional RNA Library Prep Kit (New England Biolabs), according to the manufacturer's instructions using adapters diluted 1:50. Libraries were quantitated using the Agilent High Sensitivity D1000 ScreenTape System on a 2200 Tapestation (Agilent), pooled at equimolar concentration, denatured and sequenced on a NextSeq 500 (Illumina). RNA sequencing transcript abundance was estimated from fastq files using the reference-free quantification tool salmon with the gencode GRCh37 transcript annotation. Differential expression analysis was performed using DESeq237 with treatment, time and batch (where applicable) as covariates. Geneset enrichment analysis was performed using the GSEA function from clusterProfiler, with the reactome database pathways on the gene DESeq2 Wald statistics and a minimum and maximum pathway size of 25 and 500, respectively. Pathways with a q-value <0.05 were considered significantly enriched.

For publicly-available NPC dataset where only FPKM values were available, differential expression analysis was performed using the limma package with sample groups defined through hierarchical clustering of the log 2(FPKM+1) values. Adjusted p-values were computed using the eBayes function. GSEA was performed as for DESeq2 results, but with log 2-fold change as the ranking score.

In vivo mouse experiments. The experiments have been approved by the Animal Welfare and Ethical Review Body (AWERB) of University College London according to Animals (Scientific Procedures) Act 1986 under the project license of B.V. and personal licenses of scientists involved at UCL Optimisation, growth curves and SSTR2 staining were performed in preparation of the planned experiments. 2×10⁶ C666-1 cells were subcutaneously injected in the right flank in 100 μl (50% Matrigel) of 6-8 week-old female athymic nude mice with a starting weight of around ˜20 g. Mice are kept within Home Office limits of 18-22° C. and 40-60% humidity. The mice run on a 12 h light/dark cycle that from 7 am to 7 pm. Three groups of 10 mice each were used. PEN-221 was administered at a dose level as per previous MTD study (detailed below) with PEN-221 vehicle buffer and octreotide as negative controls. Drug treatment was initiated when median tumor volume was >0.1 cm³ and mice were monitored twice weekly. Mice were scored for health, weighed and tumors were measured via calipers. Mice were culled when the humane endpoint was reached when either tumors reached >1 cm³, there were tumor ulcerations, or weight loss >20%.

Octreotide was formulated in 0.5% solutol/5% Mannitol/5 mM Acetate buffer, pH 4.0 at a concentration of equimolar to the concentration of PEN-221. Vehicle control (PEN-221 vehicle buffer) was 0.5% Solutol/5% Mannitol/5 mM Acetate buffer, pH 4.0. PEN-221: PEN-221 was formulated in 0.5% Solutol/5% Mannitol/5 mM Acetate buffer, pH 4.0 at a concentration to support dosing at 1.5 mg/kg, i.e., 0.30 mg/ml for 5 ml/kg.

Treatment was started when median tumor volume ≥0.1 cm³. Mice were allocated into three groups (1) Control: 5× weekly injection at a dosing volume of 5 ml/kg; (2) Octreotide: 5× weekly injection at a dosing volume of 5 ml/kg of the solution prepared at a concentration that is the molar equivalent to the dose of PEN-221; (3) PEN-221: 5× weekly injection at a dosing volume of 5 ml/kg of the solution prepared at an agreed upon selected dose identified from Study A above. C666-1 were xenografted and tumor specimens were stained on formalin-fixed paraffin-embedded slides as described above for SSTR2.

Patient survival analysis. IBM SPSS Statistics software (version 24) and R was used to analyze the data. The likelihood of univariable independence between groups was performed using the Pearson X2 test (and the Fisher's exact test when appropriate) for categorical variables. Rates of survival were calculated with the Kaplan-Meier method and comparison of survival by Log-rank test. SSTR2 positivity was compared using backward logistic regression analysis, also taking into account significant clinicopathological characteristics. The following clinicopathological characteristics were dichotomized: age (cutoff at 65 years), T-stage (T1/2 versus T3/4), N-stage (NO versus N1/2/3), and M-stage (MO versus M1). Two-tailed p-values <0.05 were considered statistically significant. Multivariate cox regression survival analysis was performed with the R package survival, with center (dichotomized to Europe/Asia), SSTR2 status, EBV status, sex, and age as well as T (dichotomized to T1-2 and T3-4), N (dichotomized to NO-1 and N2-3), and M staging as covariates initially. Center and sex were removed from the final model as they were found to be nonsignificant (p<0.05). The proportional hazards assumption was tested using the cox.zph function; none of the covariates in the final model were found to violate the proportional hazards assumption (p<0.05). Concordance was calculated with Cohen's Kappa.

⁶⁸Ga-DOTATATE molecular Imaging. Patients were recruited prospectively under the Pilot study of Somatostatin Receptor Imaging in Nasopharyngeal carcinoma (ClinicalTrials.gov: NCT03670342). Informed consent was obtained from all patients, and approval was obtained from the centralized institution review board (IRB protocol no. 2015/2482).

Peptide labeling with ⁶⁸Ga in the Department of Nuclear Medicine and Molecular Imaging, Singapore General Hospital was performed using an automated synthesis module (Scintomics GmbH). In brief, 40 μg DOTA-[Tyr3] Octreotate precursor (Auspep) was radiolabeled with Gallium-68 eluted from ⁶⁸Ge/⁶⁸Ga generator (iThemba, South Africa). The ⁶⁸Ge/⁶⁸Ga generator was eluted with 10 ml 0.6M hydrochloric acid. Gallium-68 was trapped using PS-H cartridge and eluted with 1.7 ml 5M sodium chloride solution into 3 ml of 1.5M HEPES buffer solution. The solution was heated at 125° C. for 6 min and purified using a Sep-Pak Light C18 cartridge and the final labelled product eluted with 2 ml of 50% Ethanol (v/v) into a product vial and diluted with 19 ml of phosphate buffered saline. Radiochemical purity was established by thin layer chromatography and the purity exceeded 90% in all cases.

Whole-body ⁶⁸Ga-DOTATATE and ¹⁸F-FDG PET/CT imaging was performed using a dedicated General Electric PET/CT system (GE Discovery 690 VCT, GE Medical Systems, LLC, Waukesha, Wisconsin USA). Scans were acquired from the skull vertex to mid-thighs. Computed tomography (CT) was performed with (for 18F-FDG PET) and without (for ⁶⁸Ga-DOTATATE PET) intravenous contrast media application for attenuation correction purposes as follows: 100 kV; “GE smart mA dose modulation” helical thickness: 3.75 mm; table speed: 0.984 mm; rotation time: 0.8 sec. Attenuation-corrected whole-body (vertex of skull to upper thighs) scans were acquired in 3-dimensional mode (2-min emission time per bed position). Depending on body length, eight bed positions were used with a field of view of 50 cm. For iterative reconstruction of the time of flight (TOF)-data, three iterations and 24 subsets with a filter cutoff of 7.0 were used. The interval between PET scans was not more than 7 days.

SSTR2 staining using UMB1 antibody (Abcam). Paraffin-embedded biopsy specimens and TMA sections of 4 μm thickness were cut and processed in an automated immunostainer (Roche Ventana, Tucson, Arizona, USA). Slides were heated to 75° C. for 8 min and deparaffinized by an EZ prep solution. Following pretreatment of the samples with EDTA at 95° C. for 16 min and subsequent addition of peroxidase inhibitor for 4 min, anti-SSTR2 antibody (rabbit monoclonal UMB1-clone (Abcam, Cambridge UK) was manually applied at 1:250 final dilution diluted in Ventana's Antibody Diluent on the sections followed by 60 min incubation at room temperature The slides were next incubated with Optiview HQ Universal Linker and Optiview HRP multimer (Ventana Medical Systems) for 8 min or with the Universal Secondary Antibody on Ventana Classic machines. The final steps included application of H₂O₂ and DAB using commercial DAB-containing Ventana kits, and counterstaining with haematoxylin. During each consecutive step of the staining process the slides were rinsed with reaction buffer. Pancreatic tissue was used as a positive control, with liver and lymphoid tissue as negative controls.

SSTR2 staining using rabbit polyclonal antibodies (BioTrend, Cologne Germany). Paraffin-embedded biopsy specimens and TMA sections of 4 μm thickness were cut, heated to 75° C. for 8 min and deparaffinized by an EZ prep solution. The next steps were pretreating the samples with EDTA at 100° C. for 16 min, addition of peroxidase inhibitor for 4 min and manual application of the primary anti-SSTR2 antibody (rabbit polyclonal antibodies (BioTrend, Cologne Germany, code SS-8000-RM) at 1:5 dilution on the sections and incubation for 32 min. The slides were next incubated with Optiview HQ Universal Linker and Optiview HRP multimer (Ventana Medical Systems) for 8 min. The final steps were application of hydrogen peroxide and DAB followed by counterstaining with haematoxylin. During each consecutive step of the staining process the slides were rinsed with reaction buffer. Pancreatic tissue was used as a positive control and liver and lymphoid tissue as negative controls.

### Example 2

### Olfactory Neuroblastoma (ONB)

This study considers the largest collection of ONB tumours and associated clinical data reported to date. The multi-center and international design of the study improves the generalizability of the following findings.

SSTR-based imaging can guide diagnosis and treatment allocation. The use of SSTR2 imaging has become the standard of care for neuroendocrine tumors (NETs), allowing for improved diagnostic sensitivity and specificity. Over the course of thirty years, this area has progressed toward the routine use of ⁶⁸Ga labelled somatostain analogs PET/CT. For thoracic and abdominal NETs, 93% sensitivity and 96% specificity of SSTR2 PET/CT has been observed. Numerous somatostatin analogs and antagonists have been developed to further improve on this imaging modality. In particular, differences in binding affinity to the various SSTR variants may play a crucial role in the selection of the appropriate radionuclide-conjugated peptide for diagnostic imaging. Furthermore, intratumor variability of SSTR expression may warrant the use of peptides with broader binding affinity in order to adequately capture the expression profile of the tumor.

Our data indicate that the vast majority of ONBs overexpress SSTR2 and that this is associated with ⁶⁸Ga-DOTATOC uptake, which warrant more extensive prospective trials on the usage of SSTR2 PET/CT for diagnosis and surveillance in this disease type. Preliminary studies used ¹¹¹In-Octreotate PET/CT in the detection of recurrent disease and extensive neck and chest metastases. Due to the variability of FDG uptake in ONB, particularly in well differentiated tumors or those with low metabolic rate, the exploitation of the high expression of SSTR2 appears to be very useful, enabling detection of recurrent disease and metastases.

A role for PRRT in metastatic disease. Importantly, overexpression of SSTR2 in ONB opens the door for the implementation of peptide receptor radionuclide therapy (PRRT) as treatment, particularly in cases of aggressive relapse and persistent disease. In a case report Savelli et al. have demonstrated the feasibility of this treatment modality, demonstrating successful detection of brain lesions upon recurrence and treatment of a patient with PRRT. Schneider et al. similarly applied PRRT for the palliative treatment of one case of refractory ONB of high Hyams grade with metastases to the lymph nodes. Four cycles of PRRT resulted in partial response from all lesions and improved symptom management. More recently, another retrospective study similarly demonstrated feasibility of PRRT with partial response in four of seven patients, two had disease stabilization and one experienced disease progression.

In order to test the efficacy of SSTR2-targeting peptide receptor radionuclide therapy for the treatment of metastatic disease, we prospectively enrolled a subgroup of our cohort in the LUTHREE trial (NCT03454763). According to the inclusion criteria the overexpression of SSTR2 in the biopsies and the association with in vivo uptake of ⁶⁸Ga-DOTATATE uptake was confirmed in all patients. The three ONB patients that were enrolled completed therapy and we observed the efficacy of PRRT in stabilizing metastatic and otherwise inoperable disease. This provides a promising option in otherwise untreatable cases and may extend survival considerably by slowing, if not halting, the progression of disease.

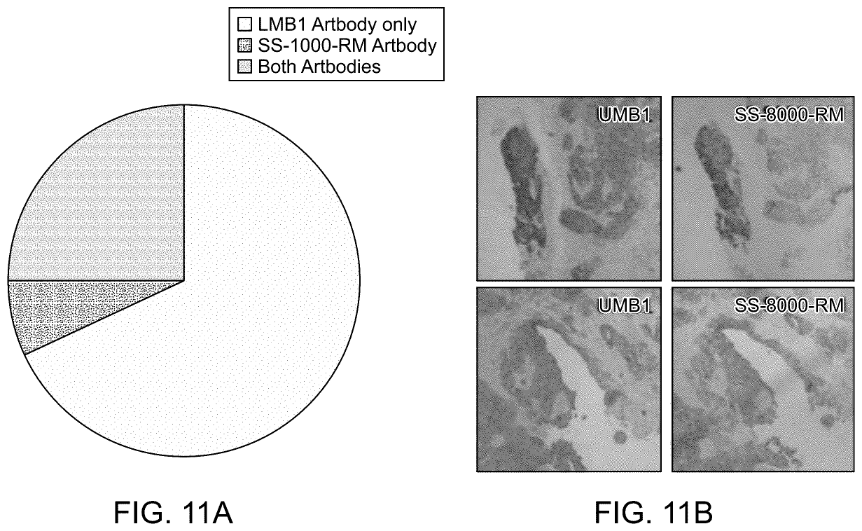

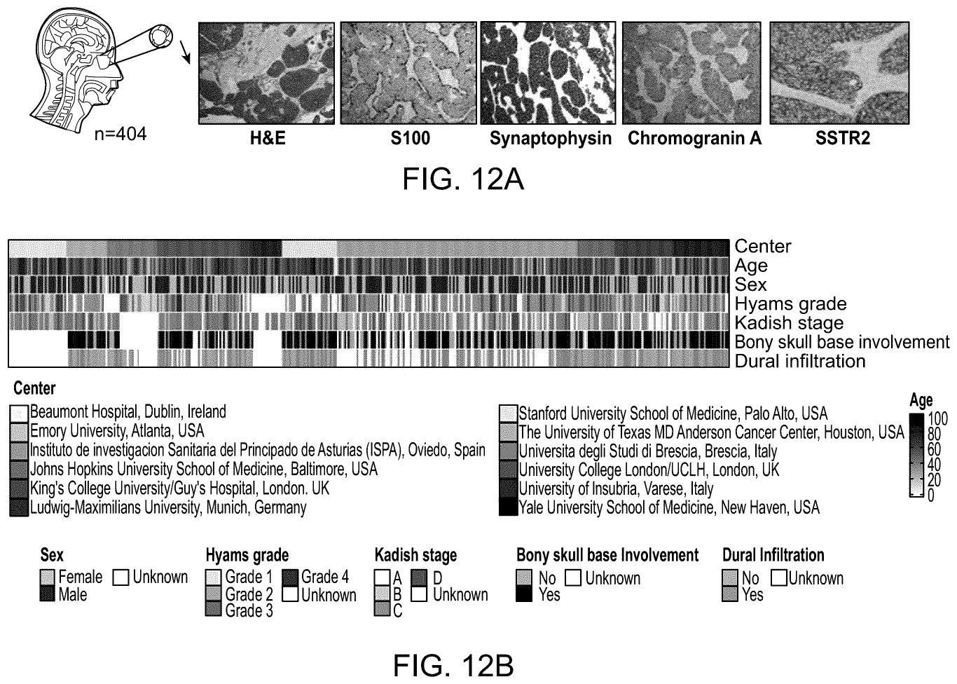

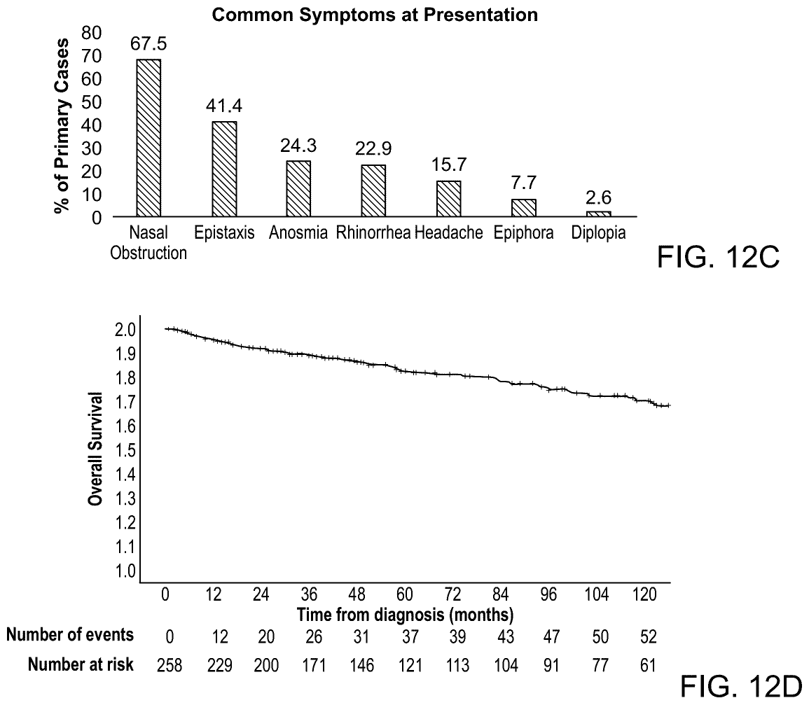

Clinical characteristics and presentation of patients with olfactory neuroblastoma. 404 cases of histologically confirmed ONB from 12 institutions in the United States and Europe (FIGS. 11A and 11B, Table 8) were analyzed. 54.1% of patients were male and 70.4% presented with primary disease (FIG. 12A). 18.0% and 8.9% presented with recurrent or persistent disease, respectively; 30.6% of patients had received prior treatment. The mean age at diagnosis for primary cases was 50.9 (range 2-91) years and, contrary to previous reports, we did not observe a bimodal age distribution, rather a single peak was observed (FIG. 12B). For patients who had not received prior treatment, typical symptoms at presentation were nasal obstruction, epistaxis, anosmia, rhinorrhea, headache, epiphora and diplopia; these were present in 67.5%, 41.4%, 24.3%, 22.9%, 15.7%, 7.7% and 2.6% of patients, respectively (FIG. 11C).

TABLE 8

U.K.

| | | | UCL/UCLH, | Guy's and St. |
|---|---|---|---|---|
| | | | London | Thomas' London |
| Total | | | 33 | 7 |
| | Sex | Male | 16 | 2 |
| | | Female | 17 | 5 |
| | | Missing | 0 | 0 |
| | Hyam's | 1 | 1 | 8 |
| | Grade | 2 | 15 | 2 |
| | | 3 | 4 | 0 |
| | | 4 | 4 | 1 |
| | | Missing | 2 | 3 |
| | Kadish-Morita | A | 9 | 2 |
| | Stage | B | 7 | 0 |
| | | C | 10 | 5 |
| | | D | 2 | 0 |
| | | Missing | 5 | 0 |
| | Dulguerov | T1 | 10 | 2 |
| | T-Stage | T2 | 11 | 0 |
| | | T3 | 1 | 4 |
| | | T4 | 4 | 1 |
| | | Missing | 7 | 0 |
| | Dural | Yes | 10 | 1 |
| | Infiltration | No | 20 | 6 |
| | | Missing | 3 | 0 |
| | Bony Skull Base | Yes | 22 | 5 |
| | Involvement | No | 8 | 2 |
| | | Missing | 3 | 0 |
| | Intracranial | Yes | 0 | 0 |
| | Involvement | No | 0 | 0 |
| | | Missing | 33 | 7 |
| | | Yes | 0 | 0 |
| | Orbital | No | 0 | 0 |
| | Movement | Missing | 33 | 7 |

| | | U.S.A. | | | | |
|---|---|---|---|---|---|---|
| | | MD | | John | | Yale |
| | | Anderson | Stanford | Hopkins | Emory | University, |
| | | Cancer | University, | University, | University, | New |
| | | Center, | Palo Alto, | Baltimore, | Atlanta, | Haven, |
| | | Houston, Tx | CA | MD | GA | CT |
| Total | | 135 | 31 | 47 | 23 | 2 |
| Sex | Male | 83 | 13 | 28 | 13 | 1 |
| | Female | 52 | 18 | 19 | 8 | 1 |
| | Missing | 0 | 0 | 0 | 2 | 0 |
| Hyam's | 1 | 2 | 0 | 8 | 2 | 0 |
| Grade | 2 | 82 | 12 | 23 | 15 | 0 |
| | 3 | 17 | 3 | 11 | 1 | 0 |
| | 4 | 10 | 0 | 5 | 0 | 0 |
| | Missing | 24 | 16 | 0 | 5 | 2 |
| Kadish- | A | 18 | 2 | 4 | 1 | 0 |
| Morita | B | 36 | 7 | 4 | 5 | 0 |
| Stage | C | 57 | 14 | 34 | 12 | 2 |
| | D | 12 | 8 | 5 | 4 | 0 |
| | Missing | 12 | 0 | 0 | 1 | 0 |
| Dulguerov | T1 | 0 | 4 | 8 | 0 | 0 |
| T-Stage | T2 | 0 | 5 | 13 | 7 | 0 |
| | T3 | 0 | 6 | 12 | 10 | 0 |
| | T4 | 0 | 15 | 10 | 5 | 0 |
| | Missing | 135 | 1 | 4 | 1 | 2 |
| Dural | Yes | 52 | 15 | 18 | 5 | 1 |
| Infiltration | No | 16 | 17 | 18 | 16 | 1 |
| | Missing | 67 | 0 | 11 | 2 | 0 |
| Bony Skull | Yes | 68 | 24 | 28 | 18 | 2 |
| Base | No | 46 | 7 | 12 | 3 | 0 |
| Involvement | Missing | 21 | 0 | 7 | 21 | 0 |
| Intracranial | Yes | 65 | 12 | 11 | 9 | 0 |
| Involvement | No | 49 | 19 | 29 | 12 | 0 |
| | Missing | 21 | 0 | 7 | 2 | 2 |
| Orbital | Yes | 29 | 6 | 3 | 4 | 0 |
| Movement | No | 83 | 25 | 39 | 17 | 0 |
| | Missing | 23 | 0 | 5 | 2 | 2 |

| | | Europe | | | | |
|---|---|---|---|---|---|---|
| | | | | | | Ludwig- |
| | | University of | Universita | Instituto | Beaumont | Maximilians |
| | | Insubria, | degil Studi | de ISPA, | Hospital, | University, |
| | | Varese, | di Brescia, | Oviedo, | Dublin, | Munich, |
| | | Italy | Italy | Spain | Ireland | Germany |
| Total | | 29 | 21 | 28 | 32 | 16 |
| Sex | Male | 11 | 11 | 11 | 20 | 7 |
| | Female | 18 | 10 | 15 | 12 | 8 |
| | Missing | 0 | 0 | 2 | 0 | 1 |
| Hyam's | 1 | 6 | 0 | 12 | 8 | 0 |
| Grade | 2 | 16 | 11 | 4 | 10 | 0 |
| | 3 | 7 | 9 | 5 | 10 | 0 |
| | 4 | 0 | 1 | 0 | 4 | 0 |
| | Missing | 0 | 0 | 7 | 0 | 16 |
| Kadish- | A | 5 | 1 | 0 | 0 | 5 |
| Morita | B | 7 | 10 | 1 | 10 | 0 |
| Stage | C | 16 | 10 | 6 | 20 | 10 |
| | D | 1 | 0 | 0 | 2 | 0 |
| | Missing | 0 | 0 | 21 | 0 | 1 |
| Dulguerov | T1 | 6 | 5 | 5 | 0 | 0 |
| T-Stage | T2 | 9 | 5 | 5 | 0 | 0 |
| | T3 | 7 | 7 | 4 | 0 | 0 |
| | T4 | 7 | 4 | 0 | 0 | 0 |
| | Missing | 0 | 0 | 14 | 32 | 16 |
| Dural | Yes | 11 | 8 | 4 | 0 | 0 |
| Infiltration | No | 18 | 13 | 11 | 0 | 0 |
| | Missing | 0 | 0 | 13 | 32 | 16 |
| Bony Skull | Yes | 19 | 16 | 4 | 0 | 0 |
| Base | No | 10 | 4 | 3 | 0 | 0 |
| Involvement | Missing | 0 | 1 | 21 | 32 | 16 |
| Intracranial | Yes | 0 | 0 | 3 | 0 | 0 |
| Involvement | No | 0 | 0 | 5 | 0 | 0 |
| | Missing | 29 | 21 | 20 | 32 | 16 |
| Orbital | Yes | 0 | 0 | 1 | 0 | 0 |
| Movement | No | 0 | 0 | 6 | 0 | 0 |
| | Missing | 29 | 21 | 21 | 32 | 16 |

Clinical information for all case, at presentation, by center.

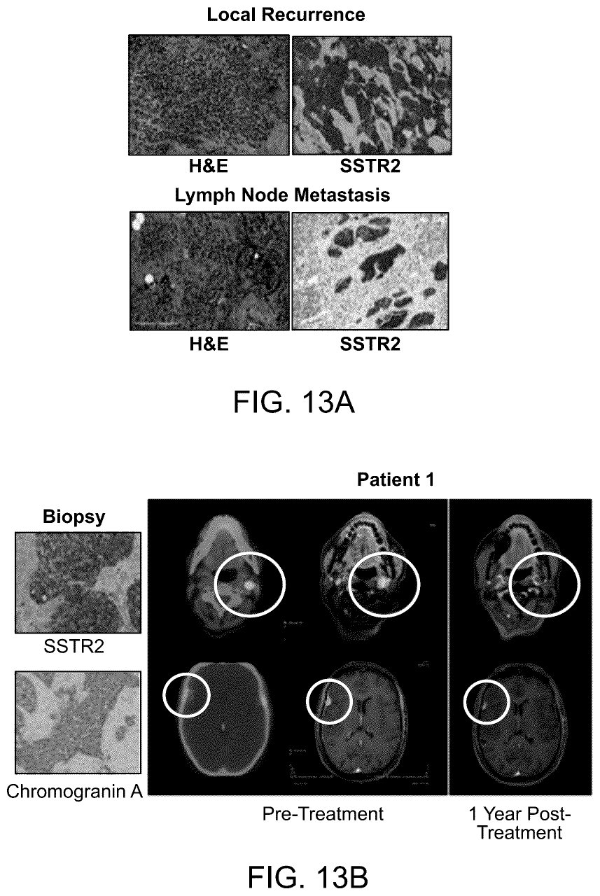

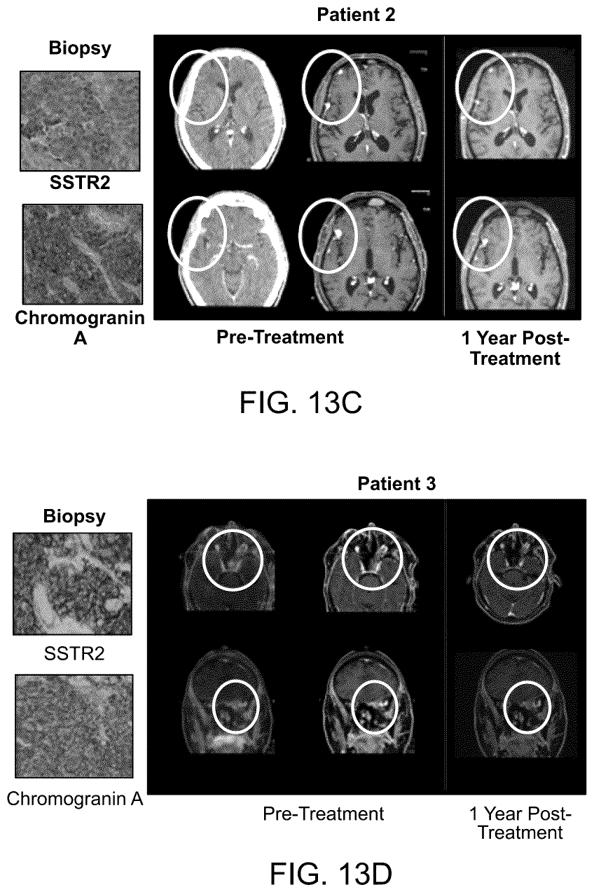

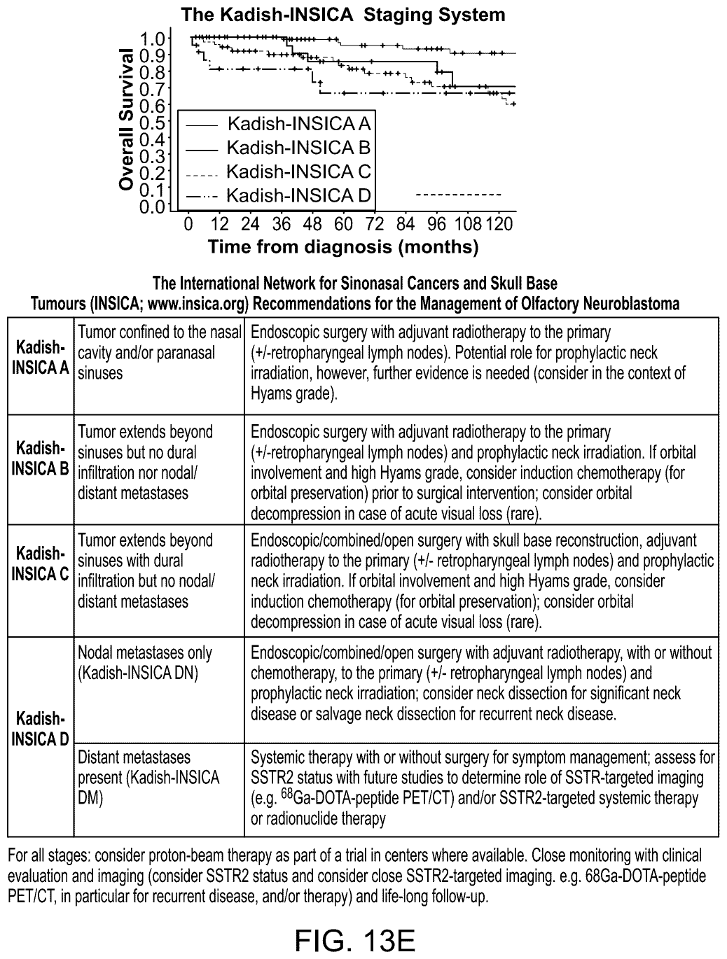

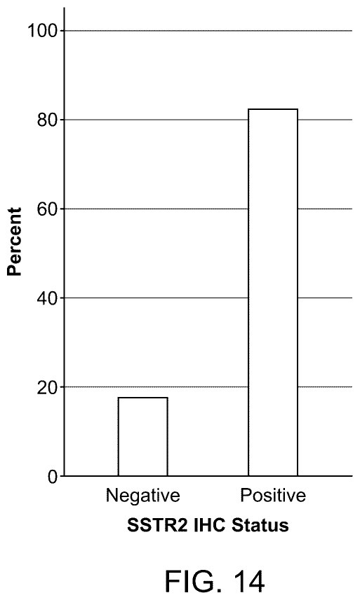

The Role of SSTR2 in the Diagnosis and Management of ONB. 82.4% of the one hundred forty-two primary tumors, for which staining was available, expressed SSTR2 by immunohistochemical assessment, however, there is no strong evidence of an association with SSTR2 expression and survival. Representative images of SSTR2 expression in recurrent and metastatic disease are demonstrated in FIG. 13.

From our cohort, three patients with histologically confirmed ONB were enrolled in the LUTHREE trial (NCT03454763). Protein expression of SSTR2 and ⁶⁸Ga-DOTA-peptide imaging demonstrate the utility of SSTR2-based imaging in the diagnosis and monitoring of disease. In these three cases, 177lu-DOTA-TATE PRRT was used for metastatic or persistent disease, after all other treatment options had been exhausted and surgery was not deemed an option. Peptide-radionuclide receptor therapy (PRRT) was well-tolerated with two cases of grade 1 neutrophils, which were self-limiting and did not interfere with the normal prosecution of therapy performed according to the trial protocol. For all three cases, PRR T stopped disease progression in the first instance. Two patients experienced relapse, 12 and 62 months after initial administration of PRRT. Of these, treatment for one is ongoing while the second has since completed re-PRRT and has stable disease. All patients are currently alive with disease.

### Methods

Patients. De-identified data on 404 ONB patients was obtained from 5 US institutions (The University of Texas MD Anderson Cancer Center, USA; Johns Hopkins University School of Medicine, USA; Stanford University School of Medicine, USA; Emory University, USA; Yale University School of Medicine, USA) and 7 European institutions (University College London/University College London Hospital, UK; Beaumont Hospital, Ireland; University of Insubria, Italy; Universita degli Studi di Brescia, Italy; Ludwig-Maximilians University, Germany; King's College/Guy's Hospital, UK; Instituto de Investigacion Sanitaria del Principado de Asturias, Spain). Inclusion criteria required confirmed histopathological diagnosis of ONB with histological characterization and sample/cohort selection performed by head and neck pathologists experienced in the evaluation of ONB. Clinical data were obtained retrospectively and reviewed by the lead team. Data collected include patient demographics, tumor status at presentation (i.e. expression of immunohistochemical markers including SSTR2, clinical stage and grade), treatment details and survival outcome. IRB approval was obtained from all institutions with further approval for multi-center data analysis from University College London IRB/Research Ethics Committee (UCL REC no. 9609/002; ML/VJL).

Diagnosis and Treatment of ONB. The date of diagnosis was defined as the date of tissue extraction for histological determination of the diagnosis. Patients were treated per their respective institution's standard-of-care and all institutions involved are tertiary level centers of excellence with long-standing experience in the diagnosis and management of this disease. In general, surgical resection with curative intent was conducted in the first instance, with or without adjuvant chemoradio- or radiotherapy. Surgery was conducted with either an endoscopic, open or combined approach.

Immunohistochemical analysis of expression of SSTR2. Immunohistochemistry was performed in different institutions, all using a standardized Ventana automated staining protocol, shared by the lead team. The rabbit monoclonal antibody UMB1 (Abcam, Cambridge, UK) was used to detect SSTR2. The slides were evaluated under the guidance of head and neck pathologists. The evaluators of the immunohistochemical stains were blinded to the clinical outcomes. The slides were dichotomously scored as being positive or negative, based on the extent of staining and intensity. The extent was scored on a continuous scale from 0%-100%. The intensity was scored as 3 categories (1: weak staining not easily seen via the low power objective; 2: moderate staining still seen on a low power objective; 3: strong staining easily visible via a low power objective), as per M. Lechner et al.

SSTR2-based PET Imaging and Peptide-Receptor Radionuclide Therapy. A subgroup of our patients with recurrent disease unsuitable for further surgery and/or radiation were recruited prospectively under the LUTHREE randomized phase II comparative study of ¹⁷⁷Lu-DOTATATE PRRT in SSTR2-positive tumors (clinicaltrials.gov: NCT03454763) and treated every 5 and 8-10 weeks. Informed consent was obtained from all patients and ethical approval was obtained (EudraCT number: 2015-004727-31). Recruitment and treatment took place at the ISRT (Istituto Scientifico Romagnolo per lo Studio e la curadei Tumori, Meldola, FC, Italy). 450 patients diagnosed with SSTR2-positive neuroendocrine tumors have been recruited to this trial in total and our subset of patients with olfactory neuroblastoma was enrolled prospectively. A diagnostic OctreoScan and ⁶⁸Ga-DOTA-peptide PET imaging were performed for each patient. Only patients with a tumor uptake scores of grade 2 or 3 were considered for therapy (the Tumor Uptake score is based on planar scintigrams obtained 24-hours post-administration of imaging and is composed of a 3-grade scale, where 1=liver uptake, 2>liver uptake and <kidney uptake and 3>kidney uptake). In every experimental arm, patients received 5 cycles of ¹⁷⁷lu-DOTATATE PRRT at 5.5 or 3.7 GBq dose. Lower dosages were administered in cases of kidney or bone marrow risk factors. ⁶⁸Ga-DOTATOC PET and MRI imaging were performed at baseline, after the third therapeutic cycle and every three months after therapy for the first two years, then every six months thereafter.

Patient survival analysis. IBM SPSS Statistics software (version 24) and R was used to analyze the data. The likelihood of univariable independence between groups was performed using the Pearson X2 test (and the Fisher's exact test when appropriate) for categorical variables. Rates of survival were calculated with the Kaplan-Meier method and comparison of survival by Log-rank test. SSTR2 positivity was compared using backward logistic regression analysis, also taking into account significant clinicopathological characteristics. The following clinicopathological characteristics were dichotomized: age (cutoff at 65 years), T-stage (T1/2 versus T3/4), N-stage (NO versus N1/2/3), and M-stage (M0 versus M1). Two-tailed p-values <0.05 were considered statistically significant. Multivariate cox regression survival analysis was performed with the R package survival, with center (dichotomized to Europe/Asia), SSTR2 status, EBV status, sex, and age as well as T (dichotomized to T1-2 and T3-4), N (dichotomized to NO-1 and N2-3), and M staging as covariates initially. Center and sex were removed from the final model as they were found to be nonsignificant (p<0.05). The proportional hazards assumption was tested using the cox.zph function; none of the covariates in the final model were found to violate the proportional hazards assumption (p<0.05). Concordance was calculated with Cohen's Kappa.

### Example 3

### SSTR2 in Brain Cancers

We have observed widespread overexpression of SSTR2 in various skull base malignancies, head and neck cancers and brain cancers, most notably in olfactory neuroblastoma. As disclosed in Example 2, we conducted a retrospective analysis of 404 primary, locally recurrent, and metastatic olfactory neuroblastoma patients from twelve institutions in the US, UK and Europe. We demonstrated SSTR2 surface expression in 82.4% of cases. We also evaluated the efficacy of managing metastatic cases with SSTR2-targeted peptide-radionuclide receptor therapy (177Lu-dotatate) as part of a larger clinical trial (LUTHREE trial; NCT03454763). In these patients, for who all other treatment options had been exhausted, further management with SSTR2-targeted therapy proved to be both well-tolerated and effective, with stable disease observed, thus demonstrating the utility of targeting this marker in this malignancy. We have also established cell culture models from olfactory neuroblastoma patients, which we have since utilized to demonstrate the antitumor efficacy of targeted therapies, including an SSTR2-targeting drug conjugate.

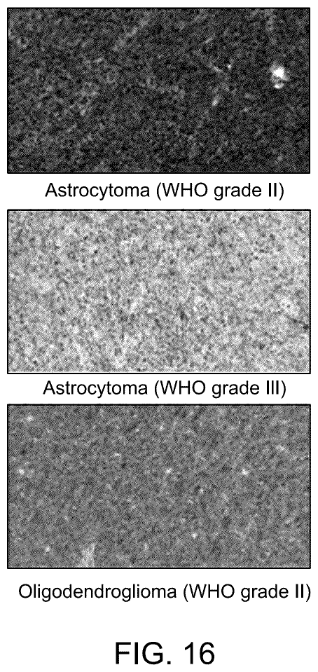

As shown in FIG. 16, there is positive SSTR2 expression in brain cancers, including astrocytoma and oligodendroglioma.

Based on these results, a multi-cancer landscape of SSTR2 expression is established by standardizing and validating an immunohistochemical method and applying it to a large dataset, comprising formalin-fixed paraffin-embedded tissue and associated clinical data, of various brain cancers (including meningioma and glioma), skull base tumours, and head and neck cancers.

### Example 4

### Automated Analysis of SSTR2 Expression

Staining will be scored digitally using an artificial intelligence-based digital image analysis pipeline. This AI workflow will enable high through-put quantitative assessment of stain intensity, which will be integrated with associated clinical and survival data to evaluate prognosis. SSTR2 status provides a useful companion diagnostic that allows patient treatment and care to be tailored in an individualized manner based on patients' tumor expression of this marker, shifting the management of these cancers to precision care.

Multiplex immunohistochemicalstaining. Multiplex immunohistochemistry is performed on the Leica BOND Rx^{m}. Briefly, tissue sections are incubated in BOND Epitope Retrieval Solution 2 for 30 minutes and stained with anti-SSTR2 antibody using the BOND Polymer Refine Detection kit with 15 minutes of antibody incubation. This is followed by incubation with additional antibodies for 15 minutes using the BOND Polymer Refine Red Detection kit, and optionally antibodies with an additional green chromogen developed using HIGHDEF green IHC Chromogen (AP) from Enzo.

Immunohistochemical Staining. Automated immunohistochemistry is performed on 3 μm sections. All automated staining using anti-SSTR2 antibodies are carried out on the Leica BOND Rx^{m}. All staining is performed using the Leica Bond Polymer Refine detection system (Leica Biosystems, DS9800) according to manufacturer recommendations.

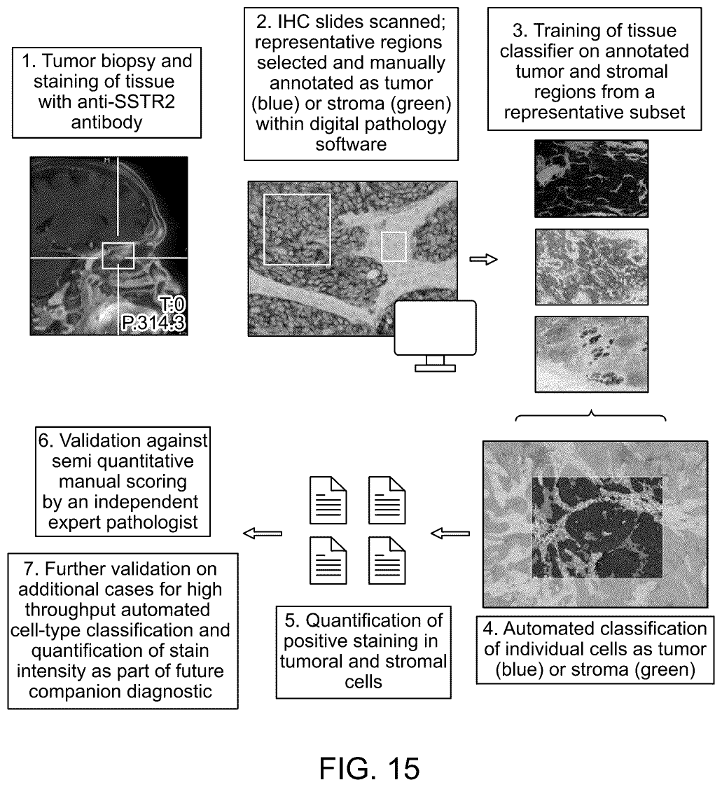

Development of QuPath Pipeline and Digital Scoring of SSTR2 Expression. The open-source digital pathology software QuPath is used to evaluate the extent of SSTR2 immunohistochemical staining in the stained cohort. A custom script is developed in the Groovy programming language. Firstly, the image is deconvolved to digitally separate the DAB and hematoxylin stains. Regions of tissue, white space, and artefact are manually annotated on a subset of sections, which is then used to train a tissue classifier. The StarDist extension, a deep-learning model, is then used to detect cell nuclei and make cell level measurements. The minimum nucleus area and minimum nucleus intensity (using the mean nuclear hematoxylin intensity) are set at 5 um2 and 0.022, respectively. The maximum nucleus area is set at 1300 um2. An object classifier is then trained on manually annotated areas of tumour and stroma and integrated into the pipeline to determine extent of DAB staining in the respective regions. The output of the analysis includes the number of cell detections and H-score. The H-score is the sum of the percentage of tumor cells stained (calculated separately), multiplied by the degree of intensity (calculated separately with values from 0-3), yielding scores within a range of 0 and 300. Thresholds for intensity are calculated using the means and standard deviations of DAB staining and applied to slides using QuPath's intensity classification function. Percentage of tumor cells stained, degree of intensity (calculated with values from 0-3) and H-score are used as the primary measures of SSTR2 staining. This is summarized in FIG. 15.

### REFERENCES

Chua, M. L. K., Wee, J. T. S., Hui, E. P. & Chan, A. T. C. Nasopharyngeal carcinoma. Lancet 387, 1012-1024 (2016).
Shanmugaratnam, K., Sobin, L. H. & World Health Organization. Histological Typing of Upper Respiratory Tract Tumors (World Health Organization, Geneva, 1978).
Chan, J. W., Parvathaneni, U. & Yom, S. S. Reducing radiation-related morbidity in the treatment of nasopharyngeal carcinoma. Future Oncol. 13, 425-431 (2017).
You, B. et al. A Phase II trial of erlotinib as maintenance treatment after gemcitabine plus platinum-based chemotherapy in patients with recurrent and/or metastatic nasopharyngeal carcinoma. Am. J. Clin. Oncol. 35, 255-260 (2012).
Gabriel, M. et al. 68Ga-DOTA-Tyr3-octreotide PET in neuroendocrine tumors: comparison with somatostatin receptor scintigraphy and CT. J. Nucl. Med. 48, 508-518 (2007).
Ricci, S. et al. Octreotide acetate long-acting release in patients with metastatic neuroendocrine tumors pretreated with lanreotide. Ann. Oncol. 11, 1127-1130 (2000).
Caplin, M. E. et al. Lanreotide in metastatic enteropancreatic neuroendocrine tumors. N. Engl. J. Med. 371, 224-233 (2014).
Schartinger, V. H. et al. (68)Ga-DOTA (0)-Tyr (3)-octreotide positron emission tomography in nasopharyngeal carcinoma. Eur. J. Nucl. Med. Mol. Imaging 42, 20-24 (2015).
Khor, L. K. et al. (68)Ga-DOTA-peptide: a novel molecular biomarker for nasopharyngeal carcinoma. Head Neck 38, E76-E80 (2016).
Unterrainer, M. et al. 68Ga-DOTATATE PET/CT reveals Epstein-Barr virus associated nasopharyngeal carcinoma in a case of suspected sphenoid wing meningioma. Clin. Nucl. Med. 43, 287-288 (2018).
Loh, K. S. et al. Somatostatin receptors in nasopharyngeal carcinoma. Virchows Arch. 441, 444-448 (2002).
Li, Y. Y. et al. Exome and genome sequencing of nasopharynx cancer identifies NF-kappaB pathway activating mutations. Nat. Commun. 8, 14121 (2017).
Chung, G. T. et al. Constitutive activation of distinct NF-kappaB signals in EBV-associated nasopharyngeal carcinoma. J. Pathol. 231, 311-322 (2013).
Lo, A. K. et al. Activation of sterol regulatory element-binding protein 1 (SREBP1)-mediated lipogenesis by the Epstein-Barr virus-encoded latent membrane protein 1 (LMP1) promotes cell proliferation and progression of nasopharyngeal carcinoma. J. Pathol. 246, 180-190 (2018).
Raab-Traub, N. Nasopharyngeal carcinoma: an evolving role for the Epstein-Barr virus. Curr. Top. Microbiol. Immunol. 390, 339-363 (2015).
Zhang, L. et al. Genomic analysis of nasopharyngeal carcinoma reveals TME based subtypes. Mol. Cancer Res. 15, 1722-1732 (2017).
Dodd, L. E. et al. Genes involved in DNA repair and nitrosamine metabolism and those located on chromosome 14q32 are dysregulated in nasopharyngeal carcinoma. Cancer Epidemiol. Biomark. Prev. 15, 2216-2225 (2006).
Cheung, S. T. et al. Nasopharyngeal carcinoma cell line (C666-1) consistently harbouring Epstein-Barr virus. Int. J. Cancer 83, 121-126 (1999).
Lin, W. et al. Establishment and characterization of new tumor xenografts and cancer cell lines from EBV-positive nasopharyngeal carcinoma. Nat. Commun. 9, 4663 (2018).
Busson, P. et al. Establishment and characterization of three transplantable EBV-containing nasopharyngeal carcinomas. Int. J. Cancer 42, 599-606 (1988).
Johnson, M. L. & Meyer, T. First in human phase 1/2a study of PEN-221 somatostatin analog (SSA)-DM1 conjugate for patients (PTS) with advanced neuroendocrine tumor (NET) or small cell lung cancer (SCLC): phase 1 results. J. Clin. Oncol. 36, 4097-4097 (2018).
White, B. H. et al. Discovery of an SSTR2-targeting maytansinoid conjugate (PEN-221) with potent activity in vitro and in vivo. J. Med. Chem. 62, 2708-2719 (2019).
Msaouel, P., Galanis, E. & Koutsilieris, M. Somatostatin and somatostatin receptors: implications for neoplastic growth and cancer biology. Expert Opin. Invest. Drugs 18, 1297-1316 (2009).
Neidle, S. Cancer Drug Design and Discovery (Elsevier/Academic Press, Amsterdam, 2014).
Hildesheim, A. & Wang, C. P. Genetic predisposition factors and nasopharyngeal carcinoma risk: a review of epidemiological association studies, 2000-2011: Rosetta Stone for NPC: genetics, viral infection, and other environmental factors. Semin. Cancer Biol. 22, 107-116 (2012).
Niedobitek, G., Agathanggelou, A. & Nicholls, J. M. Epstein-Barr virus infection and the pathogenesis of nasopharyngeal carcinoma: viral gene expression, tumour cell phenotype, and the role of the lymphoid stroma. Semin. Cancer Biol. 7, 165-174 (1996).
Yong, S. K. et al. Associations of lifestyle and diet with the risk of nasopharyngeal carcinoma in Singapore: a case-control study. Chin. J. Cancer 36, 3 (2017).
Tsao, S. W. et al. Etiological factors of nasopharyngeal carcinoma. Oral. Oncol. 50, 330-338 (2014).
Tang, X. et al. Somatostatin analogue inhibits the mobility of prostate carcinoma cells: a new therapeutic method for advanced prostate carcinoma. Int. J. Oncol. 37, 1077-1083 (2010).
Casini Raggi, C. et al. Somatostatin receptors in non-endocrine tumours. Minerva Endocrinol. 26, 149-158 (2001).
Zhang, L. et al. Gemcitabine plus cisplatin versus fluorouracil plus cisplatin in recurrent or metastatic nasopharyngeal carcinoma: a multicentre, randomised, open-label, phase 3 trial. Lancet 388, 1883-1892 (2016).
Noorlag, R. et al. Nodal metastasis and survival in oral cancer: Association with protein expression of SLPI, not with LCN2, TACSTD2, or THBS2. Head Neck 37, 1130-1136 (2015).
Choi, S. J. et al. Preparation of compact agarose cell blocks from the residues of liquid-based cytology samples. Korean J. Pathol. 48, 351-360 (2014).
Dudas, J. et al. Nerve growth factor (NGF)-receptor survival axis in head and neck squamous cell carcinoma. Int. J. Mol. Sci. 19, 1771 (2018).
Whalen, K. A. et al. Targeting the somatostatin receptor 2 with the miniaturized drug conjugate, PEN-221: a potent and novel therapeutic for the treatment of small cell lung cancer. Mol. Cancer Ther. 18, 1926-1936 (2019).
Patro, R., Duggal, G., Love, M. I., Irizarry, R. A. & Kingsford, C. Salmon provides fast and bias-aware quantification of transcript expression. Nat. Methods 14, 417-41 (2017).
Love, M. I., Huber, W. & Anders, S. Moderated estimation of fold change and dispersion for RNA-seq data with DESeq2. Genome Biol. 15, 550 (2014).
Yu, G. C., Wang, L. G., Han, Y. Y. & He, Q. Y. clusterProfiler: an R package for comparing biological themes among gene clusters. Omics 16, 284-287 (2012).
Fabregat, A. et al. The reactome pathway knowledgebase. Nucleic Acids Res. 46, D649-D655 (2018).
Ritchie, M. E. et al. limma powers differential expression analyses for RNAsequencing and microarray studies. Nucleic Acids Res. 43, e47 (2015).
Turner J H, Reh D D. Incidence and survival in patients with sinonasal cancer: a historical analysis of population-based data. Head Neck. 2012; 34:877-85.
Thompson LDR. Olfactory Neuroblastoma. Head Neck Pathol. 2009; 3:252-9.
Song X, Wang J, Wang S, Yan L, Li Y. Prognostic factors and outcomes of multimodality treatment in olfactory neuroblastoma. Oral Oncol. 2020; 103(February):1-7.
Cranmer L D, Chau B, Rockhill J K, Ferreira M, Liao J J. Chemotherapy in Esthesioneuroblastoma/Olfactory Neuroblastoma: An Analysis of the Surveillance Epidemiology and End Results (SEER) 1973-2015 Database. Am J Clin Oncol Cancer Clin Trials. 2020; 43(3):203-9.
Joshi R R, Husain Q, Roman B R, Cracchiolo J, Yu Y, Tsai J, et al. Comparing Kadish, TNM, and the modified Dulguerov staging systems for esthesioneuroblastoma. J Surg Oncol. 2019; 119(1):130-42.
Goshtasbi K, Abiri A, Abouzari M, Sahyouni R, Wang B Y, Tajudeen B A, et al. Hyams grading as a predictor of metastasis and overall survival in esthesioneuroblastoma: a meta-analysis. Int Forum Allergy Rhinol. 2019; 9(9):1054-62.
Fu T S, Monteiro E, Muhanna N, Goldstein D P, de Almeida J R. Comparison of outcomes for open versus endoscopic approaches for olfactory neuroblastoma: A systematic review and individual participant meta-analysis. Head Neck. 2016; 38:E2306-16.
Yin Z, Wang Y, Wu Y, Zhang X, Wang F, Wang P, et al. Age distribution and age-related outcomes of olfactory neuroblastoma: a population-based analysis. Cancer Manag Res. 2018; 10:1359-64.
Rimmer J, Lund V J, Beale T, Wei W I, Howard D. Olfactory neuroblastoma: A 35-year experience and suggested follow-up protocol. Laryngoscope. 2014; 124(7):1542-9.
Kadish S, Goodman M, Wang C C. Olfactory neuroblastoma-A clinical analysis of 17 cases. Cancer. 1976; 37(3):1571-6.
Morita A, Ebersold M J, Olsen K D, Foote R L, Lewis J E, Quast L M. Esthesioneuroblastoma: Prognosis and Management. Neurosurgery. 1993; 32(5):706-15.
Dulguerov P, Calcaterra T. Esthesioneuroblastoma: The UCLA Experience 1970-1990. Laryngoscope. 1992; 102(843-849).
Dulguerov P, Allal A S, Calcaterra T C. Esthesioneuroblastoma: A meta-analysis and review. Lancet Oncol. 2001; 2(11):683-90.
Joshi R R, Husain Q, Roman B R, Cracchiolo J, Yu Y, Tsai J, et al. Comparing Kadish, TNM, and the modified Dulguerov staging systems for esthesioneuroblastoma. J Surg Oncol. 2019; 119(1):130-42.
Saade R E, Hanna E Y, Bell D. Prognosis and Biology in Esthesioneuroblastoma: the Emerging Role of Hyams Grading System. Curr Oncol Rep. 2015; 17(1):1-5.
Bell D, Saade R, Roberts D, Ow T J, Kupferman M, DeMonte F, et al. Prognostic Utility of Hyams Histological Grading and Kadish-Morita Staging Systems for Esthesioneuroblastoma Outcomes. Head Neck Pathol. 2015; 9(1):51-9.
Van Gompel J J, Giannini C, Olsen K D, Moore E, Piccirilli M, Foote R L, et al. Long-term outcome of esthesioneuroblastoma: Hyams grade predicts patient survival. J Neurol Surgery, Part B Skull Base. 2012; 73(5):331-6.
Kaur G, Kane A J, Sughrue M E, Madden M, Oh M C, Sun M Z, et al. The prognostic implications of Hyam's subtype for patients with Kadish stage C esthesioneuroblastoma. J Clin Neurosci. 2013; 20(2):281-6.
Malouf G G, Casiraghi O, Deutsch E, Guigay J, Temam S, Bourhis J. Low- and high-grade esthesioneuroblastomas display a distinct natural history and outcome. Eur J Cancer. 2013; 49(6):1324-34.
Su S Y, Bell D, Hanna E Y. Esthesioneuroblastoma, neuroendocrine carcinoma, and sinonasal undifferentiated carcinoma: Differentiation in diagnosis and treatment. Int Arch Otorhinolaryngol. 2014; 18:S149-56.
Ramsay H A, Kairemo K J A, Jekunen A P. Somatostatin receptor imaging of olfactory neuroblastoma. J Laryngol Otol. 1996; 110(12):1161-3.
Dadgar H, Norouzbeigi N, Ahmadzadehfar H, Assadi M. 68Ga-DOTATATE and 18F-FDG PET/C T for the Management of Esthesioneuroblastoma of the Sphenoclival Region. Clin Nucl Med. 2020; 45(8):e363-4.
Czapiewski P, Kunc M, Gorczyhski A, Haybaeck J, Okoh K, Reszec J, et al. Frequent expression of somatostatin receptor 2a in olfactory neuroblastomas: a new and distinctive feature. Hum Pathol. 2018; 79:144-50.
Lechner M, Schartinger V H, Steele C D, Nei W L, Wu F, To K, et al. Somatostatin receptor 2 expression in nasopharyngeal cancer is induced by Epstein Barr virus infection: impact on prognosis, imaging and therapy. Nat Commun. 2021; 12(117).
Su S Y, Kupferman M E, Demonte F, Levine N B, Raza S M, Hanna E Y. Endoscopic resection of sinonasal cancers. Curr Oncol Rep. 2014; 16(2).
Gallia G L, Asemota A O, Blitz A M, Lane A P, Koch W, Reh D D, et al. Endonasal endoscopic resection of olfactory neuroblastoma: An 11-year experience. J Neurosurg. 2019; 131(1):238-44.
Abuzeid W M, Song C, Fastenberg J H, Fang C H, Ayoub N, Jerschow E, et al. Correlations between cystic fibrosis genotype and sinus disease severity in chronic rhinosinusitis. Laryngoscope. 2018; 128(8):1752-8.
Nicolai P, Battaglia P, Bignami M, Villaret A B, Delu G, Khrais T, et al. Endoscopic surgery for malignant tumors of the sinonasal tract and adjacent skull base: A 10-year experience. Am J Rhinol. 2008; 22(3):308-16.
Lund V J, Howard D, Wei W, Spittle M. Olfactory neuroblastoma: Past, present, and future?Laryngoscope. 2003; 113(3):502-7.
Nishimura H, Ogino T, Kawashima M, Nihei K, Arahira S, Onozawa M, et al. Proton-Beam Therapy for Olfactory Neuroblastoma. Int J Radiat Oncol Biol Phys. 2007; 68(3):758-62.
Nakamura N, Zenda S, Tahara M, Okano S, Hayashi R, Hojo H, et al. Proton beam therapy for olfactory neuroblastoma. Radiother Oncol. 2017; 122(3):368-72.
Song X, Huang C, Wang S, Yan L, Wang J, Li Y. Neck management in patients with olfactory neuroblastoma. Oral Oncol. 2020; 101 (December 2019):1-6.
Miller K C, Marinelli J P, Van Gompel J J, Link M J, Janus J R, Foote R L, et al. Utility of adjuvant chemotherapy in patients receiving surgery and adjuvant radiotherapy for primary treatment of esthesioneuroblastoma. Head Neck. 2019; 41(5):1335-41.
Marinelli J P, Janus J R, Van Gompel J J, Link M J, Foote R L, Lohse C M, et al. Esthesioneuroblastoma with distant metastases: Systematic review & meta-analysis. Head Neck. 2018; 40(10):2295-303.
Geijer H, Breimer L H. Somatostatin receptor PET/C T in neuroendocrine tumours: Update on systematic review and meta-analysis. Eur J Nucl Med Mol Imaging. 2013; 40(11):1770-80.
Pauwels E, Cleeren F, Bormans G, Deroose C M. Somatostatin receptor PET ligands—the next generation for clinical practice. Am J Nucl Med Mol Imaging. 2018; 8(5):311-31.
Savelli G, Bartolomei M, Bignardi M. Somatostatin receptors imaging and therapy in a patient affected by esthesioneuroblastoma with meningeal metastases. A classic example of theranostic approach. J Neurooncol. 2016; 127(3):617-9.
Thavarool S B, Muttath G, Nayanar S, Duraisamy K, Bhat P, Shringarpure K, et al. Improved survival among oral cancer patients: Findings from a retrospective study at a tertiary care cancer centre in rural Kerala, India. World J Surg Oncol. 2019; 17(1):1-7.

That which is claimed is:

1. A method for treatment of a cancer of the head and neck suspected of expressing somatostatin receptor 2 (SSTR2) in an individual, the method comprising:
a) administering an effective dose of an SSTR2 imaging agent to an individual suspected of having a tumor expressing SSTR2; and
b) quantifying the concentration of the SSTR imaging agent within the tumor using PET-CT imaging;
wherein the concentration of the imaging agent provides an indirect measurement of SSTR2 expression that is prognostic for survival, and for treatment response with a therapeutic SSTR2 agent.

2. The method of claim 1, wherein the cancer is nasopharyngeal cancer or sinonasal cancers expressing SSTR2.

3. The method of claim 1, wherein the cancer is primary, local recurrent, or metastatic.

4. The method of claim 1, wherein the cancer is nasopharyngeal cancer.

5. The method of claim 4, wherein the nasopharyngeal cancer is non-keratinizing World Health Organization (WHO) type II or Ill.

6. The method of claim 1, wherein the cancer is olfactory neuroblastoma.

7. The method of claim 1, wherein the imaging agent is a ⁶⁸Ga-dota-peptide.

8. The method of claim 7, wherein the imaging agent is one or more of ⁶⁸Ga-dodecane tetraacetic acid (DOTA)-(Tyr³)-octreotide or edotreotide (TOC), ⁶⁸Ga-DOTA-Nal3-octreotide (NOC), and ⁶⁸Ga-DOTA-(Tyr³)-octreotate (TATE).

9. The method of claim 1, wherein the individual is predicted to have a cancer expressing SSTR2 and a positive response to an SSTR2 agent if the concentration of the imaging agent is a maximized standardized uptake value (SUVmax) of 10 or more.

10. The method of claim 1, wherein if the individual is predicted to have a positive response to a therapeutic SSTR2 agent, then an effective dose of the therapeutic SSTR2 agent is administered; and if the individual is predicted to have a negative response to the therapeutic SSTR2 agent then an effective dose of gemcitabine and cisplatin is administered.

11. The method of claim 10, wherein the therapeutic SSTR2 agent is a radiolabeled conjugate of an SSTR2 ligand.

12. The method of claim 11, wherein the radiolabel is lutetium-177 or Ytrium-90.

13. The method of claim 11, wherein the SSTR2 ligand is a somatostatin analog.

14. The method of claim 13, wherein the somatostatin analog is selected from octreotide, DOTA-TATE, DOTA-NOC, DOTA-TOC, depreotide, pasireotide, and lanreotide.

15. The method of claim 10, wherein the therapeutic SSTR2 agent is a cytotoxin conjugate of an SSTR2 ligand.

16. The method of claim 15, wherein the SSTR2 ligand is a somatostatin analog.

17. The method of claim 16, wherein the somatostatin analog is selected from octreotide, DOTA-TATE, DOTA-NOC, DOTA-TOC, depreotide, pasireotide, and lanreotide.

18. A method for treatment of a cancer of the head and neck suspected of expressing somatostatin receptor 2 (SSTR2) in an individual, the method comprising:
a) obtaining a biological sample from a tumor of an individual with cancer;
b) contacting the sample with an anti-SSTR2 ligand to stain the sample; and
c) scoring the sample based on the level of anti-SSRT2 ligand staining, wherein the scoring of the sample provides a direct measurement of SSTR2 expression that is predictive of response to treatment with an SSTR2 agent and is in itself prognostic and predictive of clinical outcome of these diseases.

19. The method of claim 10, wherein the individual is predicted to have a cancer expressing SSTR2 and a positive response to an SSTR2 agent if the score of the stained biological sample is 2 or 3.

20. The method of claim 18, wherein if the individual is predicted to have a positive response to the SSTR2 agent then an effective dose of the SSTR2 agent is administered and if the individual is predicted to have a negative response to the SSTR2 agent then an effective dose of gemcitabine and cisplatin is administered.

21. The method of claim 18, wherein the scoring is performed as a program of instructions executable by computer and performed by means of software components loaded into the computer.


SEQUENCE LISTING (19 sequences)

SEQ ID NO: 1 - DNA, 21 bp, synthetic construct
  source 1..21: mol_type = other DNA
ctttcttggctatgcaggtgg

SEQ ID NO: 2 - DNA, 22 bp, synthetic construct
  source 1..22: mol_type = other DNA
gaagatgctggtgaactgattg

SEQ ID NO: 3 - DNA, 19 bp, synthetic construct
  source 1..19: mol_type = other DNA
gcacaagagggtcgaggag

SEQ ID NO: 4 - DNA, 25 bp, synthetic construct
  source 1..25: mol_type = other DNA
catagcggaggatgacataaatgac

SEQ ID NO: 5 - DNA, 20 bp, synthetic construct
  source 1..20: mol_type = other DNA
acgctgccctagaggttttg

SEQ ID NO: 6 - DNA, 20 bp, synthetic construct
  source 1..20: mol_type = other DNA
gcagaaagcagagtctggga

SEQ ID NO: 7 - DNA, 20 bp, synthetic construct
  source 1..20: mol_type = other DNA
aggacggtgtctgtggttgt

SEQ ID NO: 8 - RNA, 25 nt, synthetic construct
  source 1..25: mol_type = other RNA
atatttgaaggtatgggccatctgc

SEQ ID NO: 9 - RNA, 25 nt, synthetic construct
  source 1..25: mol_type = other RNA
ttatacacgcctctgtcattcgtgc

SEQ ID NO: 10 - RNA, 25 nt, synthetic construct
  source 1..25: mol_type = other RNA
gaggacatatcagtggtgttcagca

SEQ ID NO: 11 - RNA, 25 nt, synthetic construct
  source 1..25: mol_type = other RNA
gcgaggagctctacttgctctgcga

SEQ ID NO: 12 - RNA, 25 nt, synthetic construct
  source 1..25: mol_type = other RNA
cccaggtctggatggtattattgaa

SEQ ID NO: 13 - RNA, 25 nt, synthetic construct
  source 1..25: mol_type = other RNA
gatttcaaattgaactcctccattg

SEQ ID NO: 14 - RNA, 19 nt, synthetic construct
  source 1..19: mol_type = other RNA
gatggaaacgaccttctat

SEQ ID NO: 15 - RNA, 19 nt, synthetic construct
  source 1..19: mol_type = other RNA
gtcatgaaccacgttaaca

SEQ ID NO: 16 - RNA, 19 nt, synthetic construct
  source 1..19: mol_type = other RNA
ccctttacgtcatccctga

SEQ ID NO: 17 - RNA, 19 nt, synthetic construct
  source 1..19: mol_type = other RNA
ggagtaccctgaggctata

SEQ ID NO: 18 - RNA, 25 nt, synthetic construct
  source 1..25: mol_type = other RNA
tacatttgcgcgttcacgttggcgc

SEQ ID NO: 19 - RNA, 25 nt, synthetic construct
  source 1..25: mol_type = other RNA
agctgcaccatgctaaggctgttgt